(19) United States
(12) Patent Application Publication
(10) Pub. No.: US 2018/0099457 A1
(43) Pub. Date: Apr. 12, 2018

(54) 3D printing systems and methods

(71) Applicants: Karl Joseph Gifford, Norcross, GA (US); Tai Dung Nguyen, Fremont, CA (US); Tue Nguyen, Fremont, CA (US)

(72) Inventors: Karl Joseph Gifford, Norcross, GA (US); Tai Dung Nguyen, Fremont, CA (US); Tue Nguyen, Fremont, CA (US)

(21) Appl. No.: 15/291,038

(22) Filed: Oct. 11, 2016

(51) Int. Cl. B29C 67/00; A61M 5/31; A61M 5/315; B33Y 30/00

(52) U.S. Cl. CPC B29C 67/0085; A61M 5/3134; A61M 2207/10; A61M 5/31586; B33Y 30/00; A61M 5/31515

(57) ABSTRACT

The coupling between the plunger and the piston can be removable. The removal of the plunger can form a split syringe, which can be used for ease of transport. The split syringe can be mounted on a print head for printing using a 3D printer.

The present application claims priority from U.S. Provisional Patent Application Ser. No. 62/240,498, filed on Oct. 12, 2015 entitled: “3D printer systems and methods” (HYREL008-PRO), which is incorporated herein by reference.

### BACKGROUND OF THE INVENTION

3D printers can be used to build solid objects by printing layers by layers of building materials. The building materials can be in liquid or semi liquid form at the 3D printer head, for example, a solid material can be heated and then extruded from a 3D printer nozzle. The layers of building materials can be solidified on a substrate.

3D printer systems can use a fused filament fabrication (FFF) process (sometimes called fused deposition modeling (FDM) process) in which a filament is moved, e.g., by a filament moving mechanism, toward a heated zone. The filament can be melted, and extruded on a platform to form a 3D object. The melted filament can adhere to the walls of the heated printer head, resulting in a deformed printed lines.

It would therefore be advantageous to have advanced 3D printing systems and methods that have improved printing mechanisms.

### SUMMARY OF THE EMBODIMENTS

In some embodiments, the present invention discloses split syringes for ease of transport. The split syringe can have a removable plunger, e.g., the plunger can be removably coupled to a piston of the split syringe.

In some embodiments, the present invention discloses print head configurations for used with a 3D printing system. The print head can include a translation mechanism, together with a mounting assembly for mounting a split syringe, for printing materials in the split syringe. The print head can also be configured to accept conventional syringes.

In some embodiments, the present invention discloses 3D printing systems that can accept print head using split syringes or conventional syringes. Further the 3D printing systems can have integrated print head, thus only need to accept split syringes or conventional syringes.

### BRIEF DESCRIPTION OF THE DRAWINGS

FIGS. 1A-1C illustrate a prior art syringe according to some embodiments.

FIGS. 2A-2D illustrate a split syringe configuration according to some embodiments.

FIGS. 3A-3C illustrate flow charts for forming split syringes according to some embodiments.

FIGS. 4A-4G illustrate coupling configurations between split syringes and plungers according to some embodiments.

FIGS. 5A-5D illustrate flow charts for coupling to a split syringe according to some embodiments.

FIGS. 6A-6C illustrate secure connection configurations between a plunger and a piston according to some embodiments.

FIGS. 7A-7D illustrate configurations for secure connections between a plunger and a piston according to some embodiments.

FIGS. 8A-8F illustrate linearly secure but rotatable connection configurations between a plunger and a piston according to some embodiments.

FIGS. 9A-9D illustrate coupling configurations between a moving mechanism and a split syringe according to some embodiments.

FIGS. 10A-10C illustrate flow charts for coupling a split syringe with a moving mechanism according to some embodiments.

FIGS. 11A-11C illustrate coupling configurations between a moving mechanism and a split syringe according to some embodiments.

FIGS. 12A-12C illustrate flow charts for coupling a split syringe with a moving mechanism according to some embodiments.

FIGS. 13A-13F illustrate a process of filling a syringe barrel for printing in a 3D printer according to some embodiments.

FIGS. 14A-14B illustrate flow charts for filling syringe barrels with materials according to some embodiments.

FIGS. 15A-15C illustrate print head configurations according to some embodiments.

FIGS. 16A-16D illustrate flow charts for forming print heads according to some embodiments.

FIGS. 17A-17C illustrate print head configurations according to some embodiments.

FIGS. 18A-18B illustrate print head configurations according to some embodiments.

FIGS. 19A-19C illustrate flow charts for forming print heads according to some embodiments.

FIG. 20 illustrates a schematic of a printer for split syringe usage according to some embodiments.

FIGS. 21A-21C illustrate flow charts for split syringes in print head or printer systems according to some embodiments.

### DETAILED DESCRIPTION OF THE EMBODIMENTS

In some embodiments, the present invention discloses 3D printing systems and methods for printing using replaceable syringe, together with replaceable syringe configurations. Printing materials, such as a paste or low melting temperature solids can be loaded to a syringe. The syringe can be installed in a print head, which can include a moving mechanism for pushing on the printing materials in the syringe out a nozzle for printing on a platform. The moving mechanism can pull back on the syringe to retract the material, e.g., for precision stopping the material when not printing, e.g., stopping without oozing the material due to the high pressure in the syringe. A heater mechanism can be included to heat the printing material.

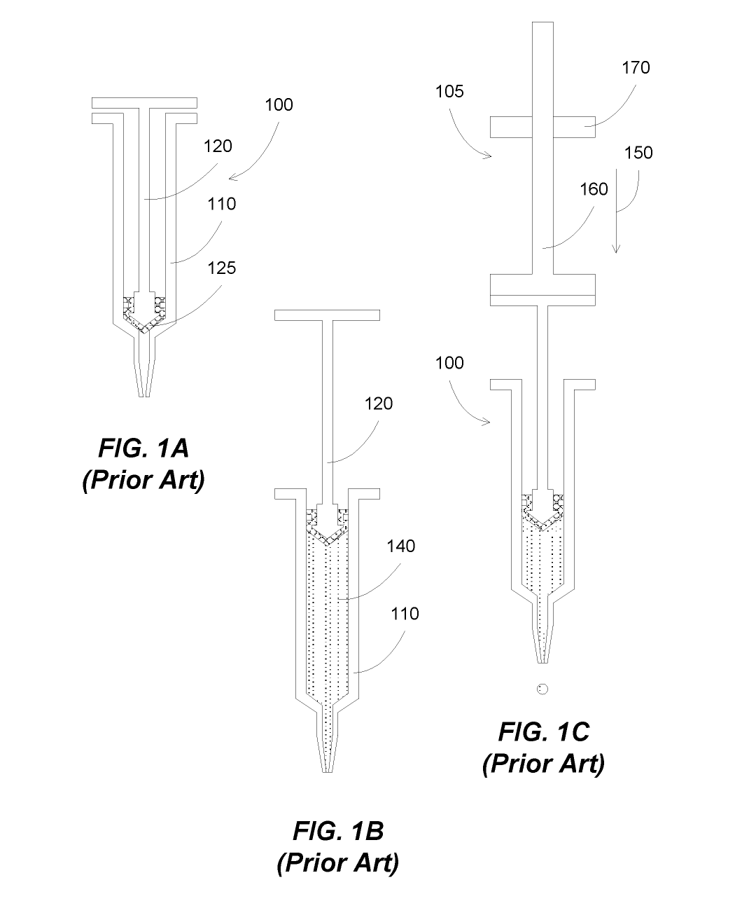

FIGS. 1A-1C illustrate a prior art syringe according to some embodiments. In FIG. 1A, an empty syringe 100 is shown in storage configuration. The syringe 100 can include a syringe barrel 110 and a plunger 120. The plunger 120 can have a plunger seal 125, which is fitted in the hollow interior of the syringe barrel 110. In the storage configuration, the plunger 120 is pushed toward the tip of the barrel 110, allowing the plunger to stay inside the barrel, thus reducing the storage or shipping volume of the empty syringe.

FIG. 1B shows the syringe filled with material 140. The plunger 120 is pulled back, for example, to the open end of the barrel, with the material 140 stored in the interior of the barrel 110. FIG. 1C shows a dispensing mechanism 105 for delivering material 14 in the syringe 100. The dispensing mechanism 105 can include a moving rod 160 and a stationary component 170. When the moving rod 160 moves downward with respect to the stationary component 170, the moving rod push on the plunger, and deliver material from the syringe.

In some embodiments, the present invention discloses split syringes and methods to use split syringes, including printing with split syringes. A split syringe can have the plunger replaced with a coupling, such as a piston. During operation, a plunger can be coupled to the coupling. An advantage of the split syringe is the shorten form factor of the split syringe, particularly with filled syringe. The split syringe can simplify packaging and shipping, since the filled split syringe can have a same volume as the empty split syringe, and can occupy less space than an empty conventional syringe.

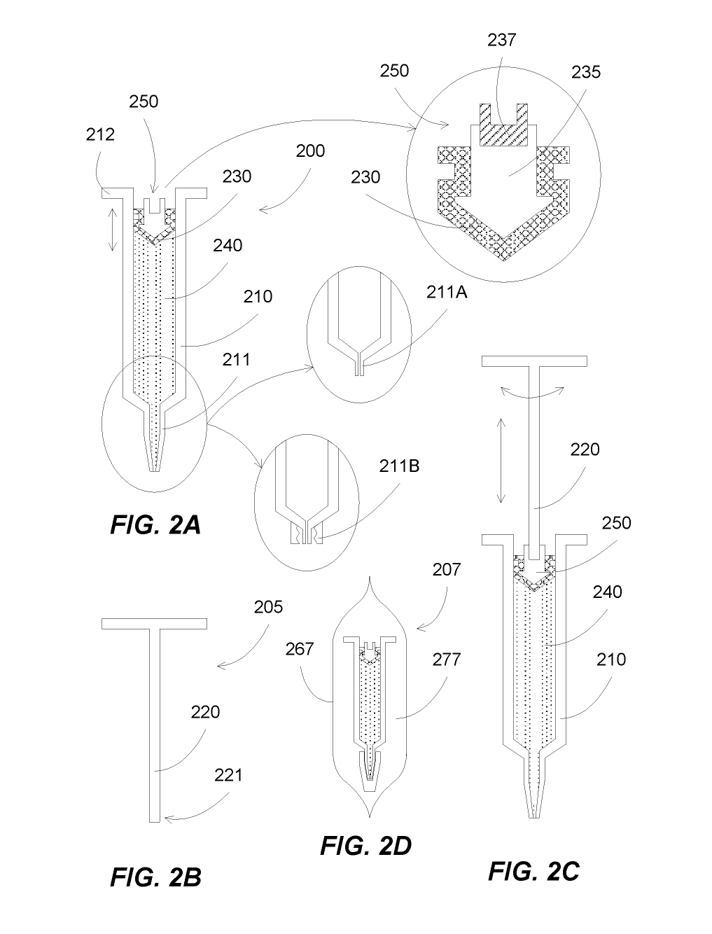

FIGS. 2A-2D illustrate a split syringe configuration according to some embodiments. FIG. 2A shows a split syringe 200. FIG. 2B shows an optional plunger 205. FIG. 2C shows an assembly configuration in which a plunger is assembled with a split syringe.

A split syringe 200 can include a hollow barrel 210. The barrel 210 can include a tip or a nozzle 211 at one end for dispensing materials, and an optional barrel handle 212 at an opposite end for support such as when an operator holding the syringe for dispensing. The barrel 210 can have different tip configurations. For example, tip 211 can include a small nozzle for dispensing material. Tip 211A can include a coupling connection for coupling, for example, with a needle by press fit, e.g., a needle can be pressed on the extruded tip of the barrel. Tip 211B can include a coupling connection for coupling, for example, with a needle by screwing, e.g., a needle can be screwed on the extruded tip of the barrel. The connection can have a taper configuration for a leak free coupling. For example, the coupling connection can include a Luer lock, e.g., a twisted and taper fitting for making a coupling with a needle. The barrel can be made from materials such as polymer-based materials, metals, alloys, glass, or ceramics. In some embodiments, the barrels can be similar to barrels of conventional syringe.

The split syringe 200 can include a piston 250 which can include a piston 237. The piston is configured to accept a plunger, e.g., a rod-like element for pushing and/or pulling on the material in the barrel 210. The piston is configured to form a movable seal with the interior of the barrel. The movable seal can move along the length of the barrel, and can push or pull the material without or with minimum leakage. For example, the piston 250 can include a body 235, which can include a piston 237 for coupling with a plunger 205, such as the coupling can be configured to couple to the connector end 221 of a plunger body 220 of the plunger 205. The coupling can be an integral part of the piston, or the coupling can be an external part that attaches to the piston. The piston 250 can include a seal 230, which conforms to the inner surface of the barrel. In some embodiments, the seal 230 can be similar to plunger seals of conventional syringe.

In some embodiments, the piston can be disposed inside the barrel, such as disposed completely insode the barrel, meaning the length of the piston is much shorter than the length of the barrel, such as shorter than half or one third of the barrel length. The piston can be configured for a tight fitting in the barrel that allows retracting and expelling a material in the barrel through the nozzle. For eample, the piston can include a rubber seal. The piston can include a short shaft, such as sorter than half, third or fourth of the barrel.

In some embodiments, the coupling between the plunger and the piston can be such that the material inside the barrel can be expelled out of the barrel through the nozzle when the plunger or the piston moves inward, e.g., toward the interior of the barrel. This action can deliver the material to a platform, such as a platform of a 3D printer, for printing.

The coupling between the plunger and the piston can be such that the material inside the barrel can be retracted toward the barrel when the plunger or the piston moves outward, e.g., out of the barrel and opposite of moving inward. This action can pull the material back to the barrel, especially at the exposed tip of the nozzle.

To print, the plunger can move inward, e.g., pushing into the barrel. To stop printing, the plunger can stop. However, the material can still be oozing out, e.g., pushed out of the barrel, for a short time after the plunger has stopped, due to the high pressure inside the barrel. This can affect the precision of the printing process. Thus the plunger can move outward, e.g., pulling out of the barrel, to reduce the high pressure and stopping the oozing of the material. To accommodate the movement of the piston in both direction, the coupling between the plunger and the piston can be rigid in the linear directions, e.g., when the plunger moves in such a way as to advance linearly into and out of the barrel, the piston can follow the movements.

For example, the plunger coupling, e.g., the coupling between the plunger and the piston, can be rigid in all directions, such as when the plunger is fixedly coupled to the piston. With such coupling, when the plunger move linearly into and out of the barrel, the piston duplicates the linear movements.

The plunger coupling can be rigid in linear directions, and rotatingly-free, such as when the plunger is coupled to the piston through a ball bearing. The plunger can be fixedly coupled to the inner ring of the ball bearing. And the piston can be fixedly coupled to the outer ring of the ball bearing. With such coupling, when the plunger move linearly into and out of the barrel, the piston duplicates the linear movements. When the plunger rotates at a same linear location, the piston can stay stationary. When the plunger rotates while advancing, such as in a spiral rotation, the piston can stay also advance at a same linear rate. Thus the plunger and the piston can form a translation mechanism, which can transform a rotation of the plunger into a linear motion of the piston.

The split syringe can be filled with a printing material 240. The piston can be at the opening of the barrel, e.g., near the barrel flange 212. For example, a plunger 205 can be connected to the piston, for example, at the coupling 237. The plunger can pull back, bringing material 240 to the inside of the barrel 210. The plunger can then be removed, e.g., disconnected from the piston. The filled split syringe can have the form factor of the barrel, which can be smaller than the empty conventional syringe.

FIG. 2D shows a package configuration for the split syringe. The split syringe 207 can be filled with a material, such as a material for used in a 3D printer, and optionally with the tip capped. The split syringe can be placed in a container 267, such as a box or a bag. The container can be sealed against the outside ambient, thus the sealed container can protect the material in the split syringe from being damaged, such as drying by exposed to outside ambient, or contaminated by the ambient contaminants. The volume 277 in the sealed container can be vacuum, e.g., air can be evacuated before sealing the container, further providing protection against damages.

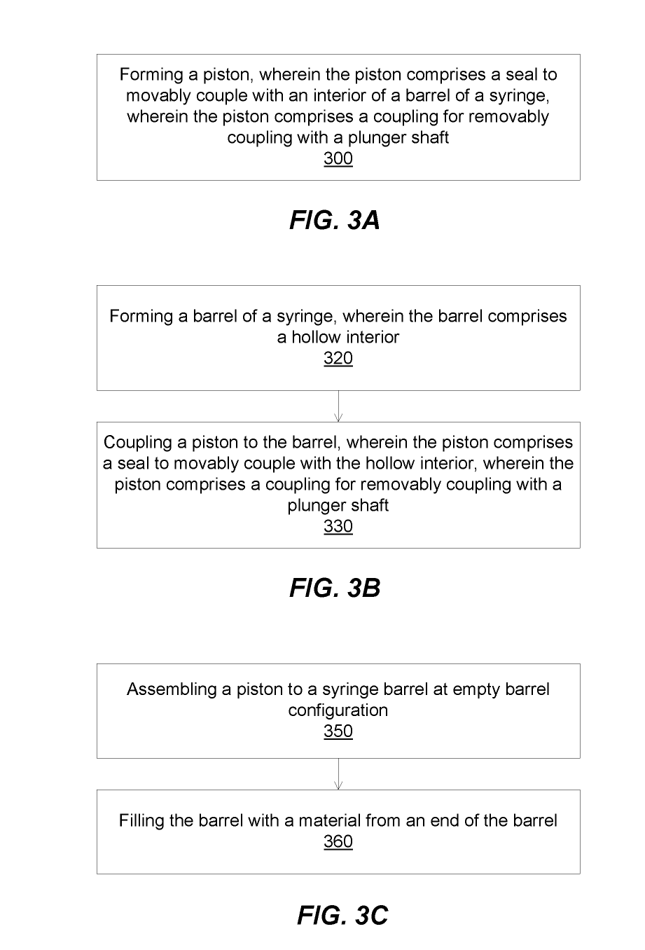

FIGS. 3A-3C illustrate flow charts for forming split syringes according to some embodiments. FIG. 3A shows methods to form pistons, which can be used with conventional syringe barrel to form split syringes. The piston can include a seal for movably sealing with the interior of the syringe barrel. The piston can include a coupling, which can be configured to coupled with a plunger, e.g., a rod-like component that can be used for pushing or pulling on materials in the syringe barrel. Operation 300 forms a piston, wherein the piston can include a seal to movably couple with an interior of a barrel of a syringe. The piston can include a coupling for removably coupling with a plunger shaft. The coupling can be an integral part of the piston, or an external part that coupled to the piston. The coupling is removable, meaning the plunger can be coupled with the piston, and the plunger can be removed from the plunger/piston assembly.

The coupling can be a fixed coupling, meaning the plunger and the piston, after being coupled, can form a solid connection. For example, the plunger can include a thread, to be screwed in a thread of the piston. Thus the plunger and the piston can move as one element, such as pushing in or pulling out of the barrel to deliver or retract the material. A locking mechanism can be included to secure the plunger with the piston. For example, the locking mechanism can include a locking washer, which can secure the plunger with the piston after a thread connection.

The coupling can be a linearly fixed coupling, meaning the plunger and the piston can form a solid connection in an axial direction of the plunger, e.g., the direction of pushing in or pulling out of the barrel to deliver or retract the material, such as the direction parallel to the long length of the plunger or the barrel. For example, the coupling can include a latch, which can secure the plunger from pulling out of the piston. If the coupling is also fixed in other directions, the coupling can be a fixed coupling as discussed above. If the coupling is not fixed in a rotational direction, the coupling can be a rotation free coupling as discussed below.

The coupling can be a rotation free coupling, meaning the plunger can be freely rotated relative to the piston. For example, the coupling can include a ball bearing, with the plunger fixedly coupled to an inner (or outer) ring of the ball bearing and the piston fixedly coupled to the other ring (outer or inner ring, depending on the coupling of the plunger). The plunger can thus freely rotate while the piston remains stationary. since the plunger and the piston are fixedly coupled to the ball bearing, the coupling can be a linearly fixed coupling, e.g., the plunger and piton can move as one element in the directions of pushing or pulling materials in the barrel.

The rotation free and linearly fixed coupling can function as a translation mechanism, e.g., converting a rotation motion of the plunger into a linear motion of the piston. For example, if the plunger rotates while advancing in a linear direction, such as moving in a spiral motion, the piston can advance in the linear motion. The translation mechanism can push out and pull in the material in the barrel, forming a print head assembly for a 3D printer.

FIG. 3B shows methods to form split syringes, which includes forming a syringe barrel and a piston having a coupler for coupling with a plunger. The method can include optionally assembling the piston in the syringe barrel. Operation 320 forms a barrel of a syringe, wherein the barrel can include a hollow interior. Operation 330 couples a piston to the barrel, wherein the piston can include a seal to movably couple with the hollow interior. The piston can include a coupler for removably coupling with a plunger shaft. A piston can be formed independently from the syringe barrel.

The coupler can be an integral part of the piston, or an external part that coupled to the piston. The coupling between the coupler and the plunger can removable, meaning the plunger can be coupled with the piston, and the plunger can be removed from the plunger/piston assembly. The coupling can be a fixed coupling, a linearly fixed coupling, a rotation free coupling, or a combination of linearly fixed coupling and rotation free coupling.

FIG. 3C shows methods to form filled split syringes, which can include filling the split syringe from the tip end, pushing the piston from the tip end toward the barrel flange side of the syringe barrel. Operation 350 assembles a piston to a syringe barrel at empty barrel configuration. For example, a piston can be placed inside the barrel of a syringe, at the opening of the barrel, e.g., at the barrel flange end of the barrel. The piston can be pushed toward the tip end of the barrel. The tip of the barrel can be open, to allow air to escape the barrel. Thus there can be minimum air volume in the barrel. Operation 360 fills the barrel with a material from an tip end of the barrel. For example, a second syringe can be coupled to the tip of the split syringe, e.g., to the tip of the barrel. The second syringe can be filled with the material, which can be transferred to the split syringe, for example, by pushing the plunger of the second syringe.

Alternatively, the material can be pushed to the barrel from the barrel flange end. Then the piston can be placed in the barrel, capping the material.

Alternatively, a plunger can be coupled to the piston which can be disposed in the syringe barrel. The plunger can push the piston to the tip end, e.g., emptying the air in the barrel. The tip of the split syringe can be placed in contact with the material. For example, if the material is a fluid, the tip of the split syringe can be dipped in the fluid. If the material is a semi-solid or a paste, the tip of the split syringe can be coupled with a source of the material, which is under pressure. Thus by pulling on the plunger, the material can be transferred to the barrel of the split syringe. Since the material is under pressure, the pressure can assist in pushing the material from the source of material to the barrel.

In some embodiments, the material in the barrel can be without any bubbles. Since the split barrel can be used in a 3D printer for printing objects, any void in the syringe can result in a defect in the printed objects. Thus the filling of the syringe with the material can be perform to prevent any voids or bubbles in the material.

For example, the material can be slowly filled in the barrel to avoid bubbles. When the piston is placed in the barrel, the piston should contact the material and pushing the material out of the barrel when the piston is moving into the barrel, to avoid trapping air in the barrel.

Also vacuum process can be used to remove bubbles. The syringe, after filled with the material, can be placed in a vacuum chamber. For example, the syringe can be placed with the nozzle exposed and placed upward. Thus there can be a vacuum surface at the exposed nozzle opening, which can attract bubbles in the material. Alternatively, the piston can be removed or not placed in the barrel of the syringe. The syringe then can be placed in the vacuum chamber with the flange end (e.g., the end of the barrel that is configured to place the piston and the plunger to the barrel, which can be the opposite end of the nozzle) exposed and upward. Thus there can be a vacuum surface at the exposed barrel opening at the flange end, which can attract bubbles in the material. The opening at the flange end can be larger than the opening of the nozzle, thus the vacuum bubble removal process can be faster.

In some embodiments, the present invention discloses coupling configurations between split syringes and plungers, e.g., rod-like elements that can be configured to deliver materials in the split syringes.

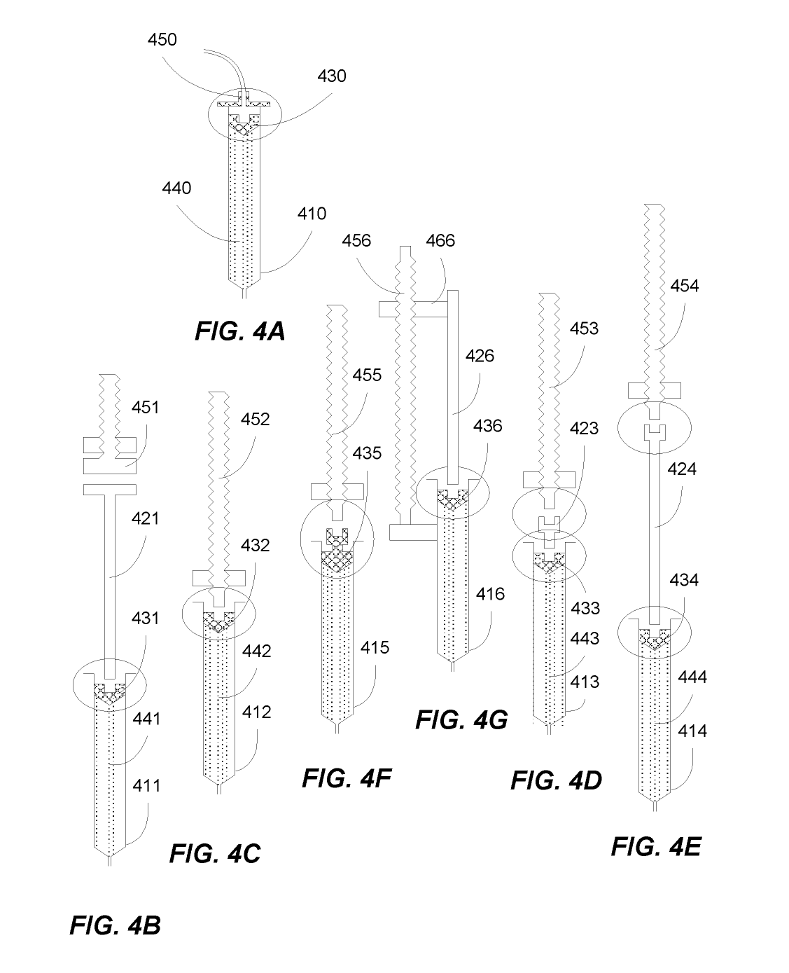

FIGS. 4A-4G illustrate coupling configurations between split syringes and plungers according to some embodiments.

In FIG. 4A, a pneumatic or hydraulic pressure con be used as a plunger for delivering materials from the split syringe. A coupling 450 can allow air or fluid to enter the split syringe 410. The air or fluid can be under pressure, which can push the material 440 from the split syringe outward, for example, through the force action on the piston 430. In addition, releasing the pressure, or exerting a negative pressure (such as a suction action) can pull the material 440 back into the split syringe.

In FIG. 4B, a plunger 421 can be coupled to a piston 431 of a split syringe 411. A lead (or ball) screw mechanism 451 can be used to push the plunger 451, delivering material 441 from the split syringe 411. The lead screw mechanism 451 can be fixed attached to the plunger 421, thus can revert the direction and pulling material 441 back into the split syringe. The coupling between the lead screw mechanism 451 and the plunger 421 can be a rotation free coupling, which can allow the lead screw to rotate while not rotating the piston.

In some embodiments, a regular syringe can be used, instead of a split syringe with a separate plunger.

In FIG. 4C, a lead (or ball) screw mechanism 452 can be directly coupled to the piston 432 of the split syringe 412. The lead screw mechanism 452 can be actuated, which can push the piston 432, delivering material 442 from the split syringe 412. The lead screw mechanism 452 can revert the direction and pulling material 442 back into the split syringe. The coupling between the lead screw mechanism and the piston can be a rotation free and linearly fixed coupling, e.g., the lead screw mechanism functions as a translation mechanism, translating a rotating motion of the lead screw into a linear motion of the piston.

In FIG. 4D, an additional coupler 423 can be coupled to the piston 433 of the split syringe 413. A lead (or ball) screw mechanism 453 can be coupled to the coupler 423, and which can be used to push the coupler 423, delivering material 443 from the split syringe 413. The lead screw mechanism 453 can revert the direction and pulling material 443 back into the split syringe. At least one of the coupling between the lead screw mechanism and the additional coupler, and the coupling between the additional coupler and the piston, is rotation free and linearly fixed coupling, for example, to form a translation mechanism.

In FIG. 4E, a long coupler 424 can be coupled to the piston 434 of the split syringe 414. A lead (or ball) screw mechanism 454 can be coupled to the long coupler 424, and which can be used to push the coupler 424, delivering material 444 from the split syringe 414. The lead screw mechanism 454 can revert the direction and pulling material 444 back into the split syringe. At least one of the coupling between the lead screw mechanism and the long coupler, and the coupling between the long coupler and the piston, is rotation free and linearly fixed coupling, for example, to form a translation mechanism. An advantage of using the long coupler 424 can include the use of large lead screw, as compared to the interior volume of the split syringe barrel, since the part that enters the split syringe is the long coupler, and not the lead screw.

In FIG. 4F, a lead (or ball) screw mechanism 455 can be directly coupled to the piston 435 of the split syringe 415. The piston can include a short shaft for making a coupling with the lead screw mechanism. Alternatively, these figures show examples of female/male connections, but other connections can be used, such as male female/connections. For example, the piston has a female connection, to be mated with a male connection of the lead screw mechanism, the plunger, or the coupler. Other configurations can be used, such as a male connection for the piston, and a female connection for the lead screw mechanism, the plunger, or the coupler.

In FIG. 4G, a lead (or ball) screw mechanism 456 can be coupled to a plunger 426 through a coupler 466, such as a lead screw nut 466. When the shaft of the lead screw mechanism turns, the lead screw nut 466 can linearly move up and down. The plunger 426 can be coupled to a piston 436 of the split syringe 416. The lead screw mechanism 456 can be actuated, which can push the plunger 426 and the piston 436, delivering material 446 from the split syringe 416. The lead screw mechanism 456 can revert the direction and pulling material 446 back into the split syringe. The coupling between the lead screw mechanism and the plunger/piston can be a translation mechanism, translating a rotating motion of the lead screw into a linear motion of the plunger/piston.

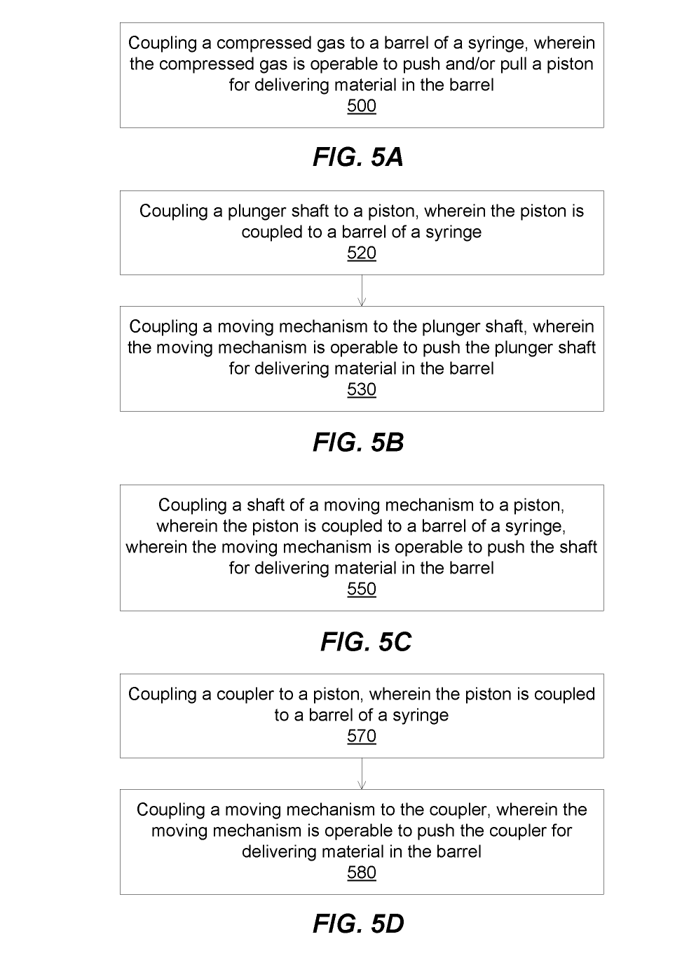

FIGS. 5A-5D illustrate flow charts for coupling to a split syringe according to some embodiments. In FIG. 5A, compressed fluid, such as compressed gas or compressed air, can be used to actuate a split syringe. Operation 500 couples a compressed gas to a barrel of a syringe, wherein the compressed gas is operable to push a piston for delivering material in the barrel. A vacuum can also be connected to the barrel for reduce the pressure, effectively pulling the material back to the syringe.

In FIG. 5B, a plunger shaft can be used between a moving mechanism, such as a lead or ball screw mechanism, to actuate a split syringe. Operation 520 couples a plunger shaft to a piston, wherein the piston is coupled to a barrel of a syringe. Operation 530 couples a moving mechanism to the plunger shaft, wherein the moving mechanism is operable to push or pull the piston, through the plunger shaft, for delivering material in the barrel. For example, the plunger shaft can move linearly, or can move spirally.

In FIG. 5C, a shaft of a moving mechanism, such as a lead or ball screw mechanism, can be coupled to a split syringe to actuate the split syringe. Operation 550 couples a moving mechanism to a plunger, wherein the plunger is coupled to a piston, wherein the piston is coupled to a barrel of a syringe, wherein the moving mechanism is operable to push or pull the piston for delivering material in the barrel. The moving mechanism can rotate the plunger, and the rotation action of the plunger can be translated to a linear motion of the piston. The moving mechanism can include a lead screw shaft coupled to a lead screw nut. The rotation of the lead screw shaft can linearly move the nut, which is fixedly coupled to the plunger, which is fixedly coupled to the piston. The rotation action of the lead screw shaft can be translated to a linear motion of the piston.

In FIG. 5D, a coupler, short or long, can be used between a moving mechanism, such as a lead or ball screw mechanism, to actuate a split syringe. Operation 570 couples a coupler to a piston, wherein the piston is coupled to a barrel of a syringe. Operation 580 couples a moving mechanism to the coupler, wherein the moving mechanism is operable to push the coupler for delivering material in the barrel.

In some embodiments, the present invention discloses split syringes having improved coupling configurations for the piston. The couplings can form a secure connection between a piston of the split syringe and a plunger, e.g., a rod-like element that can be used for pushing material out of the split syringe or pulling material back to the barrel of the split syringe. The secure connection can prevent the plunger from being detached from the piston in a pushing in or a pulling out action. Further, in some embodiments, the secure connection can allow the plunger to rotate relative to the piston.

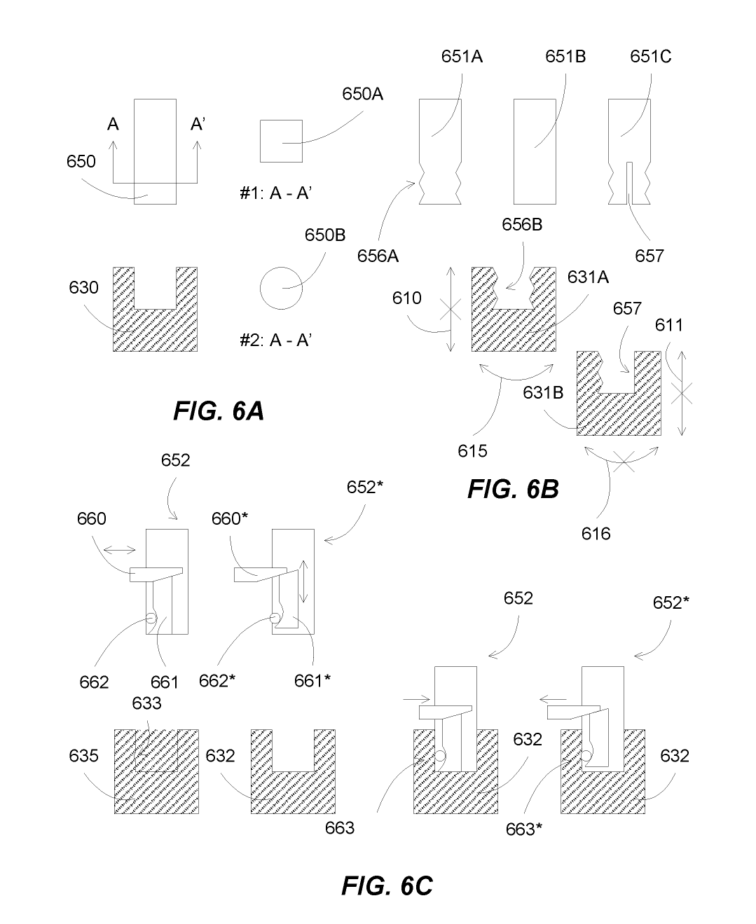

FIGS. 6A-6C illustrate secure connection configurations between a plunger and a piston according to some embodiments. The secure connection configurations can prevent the plunger, after making the connection with the piston, from being removed from the piston when the plunger is exerting a pulling action, for example, to bring the material back to the split syringe barrel.

In FIG. 6A, a press fit can be used, e.g., there can be a tight tolerance between the plunger 650 and the piston 630. The plunger can have different cross sections, such as square 650A, or circular 650B. If the connection portions of the plunger and/or the piston is made of a non-hardened material, such as a polymer or a plastic material, the plunger can have similar or slight larger dimensions than the piston, in order to form a press fit connection.

In FIG. 6B, a locking mechanism can be included to form a secure connection. The plunger can include a locking configuration 656A, and the piston can have a matching locking configuration 656B, so that when the plunger is coupled with the piston, the locking configuration can form a secure connection, preventing the plunger from being pushing in or from being easily pulling out 610 of the piston. In some embodiments, the locking mechanism can be provided on both components of the plunger 651A and the piston 631A. Alternatively, the locking mechanism can exist only in one component, such as only on the piston 631A, mating with the plunger 651B, or only on the plunger 651A and not on the piston (not shown). In some embodiments, splits can be included, either on the plunger 651C (split 657), on the piston, or on both the plunger and the piston, for example, to facilitate the formation of the secure connection. The split can allow the material to deform, e.g., the plunger to be squeezed inward to become smaller for ease of fitting into the piston, or the piston to be extended outward by become larger for ease of insertion of the plunger.

In some embodiments, the locking configuration can be continuously rotationally symmetric, e.g., the locking configuration is the same for all radial directions. This can allow the plunger 651A and the piston 631A to be rotation free, for example, the plunger can be rotated while the piston remains stationary, and vice versa. The rotation free connection can be used in a translation mechanism, such as in a lead screw mechanism with the plunger coupled to a lead screw shaft of the lead screw mechanism. Thus when the lead screw shaft of the lead screw mechanism rotates, the piston can move linearly without rotating.

The locking configuration can be asymmetric, for example, a flat 657 can be formed on a side of the piston 631B. This can fixedly coupled the plunger (651A, 651B, or 651C) with the piston 631B, e.g., there is no movement in linear directions 611 or in rotational directions 616. The rigid connection can be used in a translation mechanism, such as in a lead screw mechanism with the plunger coupled to a screw nut of the lead screw mechanism. Thus when the lead screw shaft of the lead screw mechanism rotates, the lead crew nut can move linearly, bringing the plunger and the piston with it.

In FIG. 6C, a snap-on like mechanism can be used, which can include a component, such as a ball 662, to move inward 662 (to facilitating insertion of the plunger 652 to the piston 632) and outward 662* (so that the plunger 652* can press against the wall of the piston 632 to form a secure connection). A level 660/661 can be included to actuate the movement of the ball 662. For example, a handle 660 can be pushed in, for example, by an operator. The handle 660 can push a level 661 downward, forming a room for the ball 662 to move inward, e.g., not protruding outward 663 from the wall of the plunger 652. When the handle 660* is released, a spring (not shown) can push the handle 660* out, moving the level 661* upward (for example, with the assistance of a not-shown spring). The ball 662* can be pushed out, e.g., protruded from the wall of the plunger 652*, and in the presence of the piston, pressing 663* against the wall of the piston 632 to form a secure connection between the plunger 652* and the piston 632.

The piston 632 can have flat vertical walls. The piston 635 can have one or more notches on the vertical walls to accommodate the ball 662.

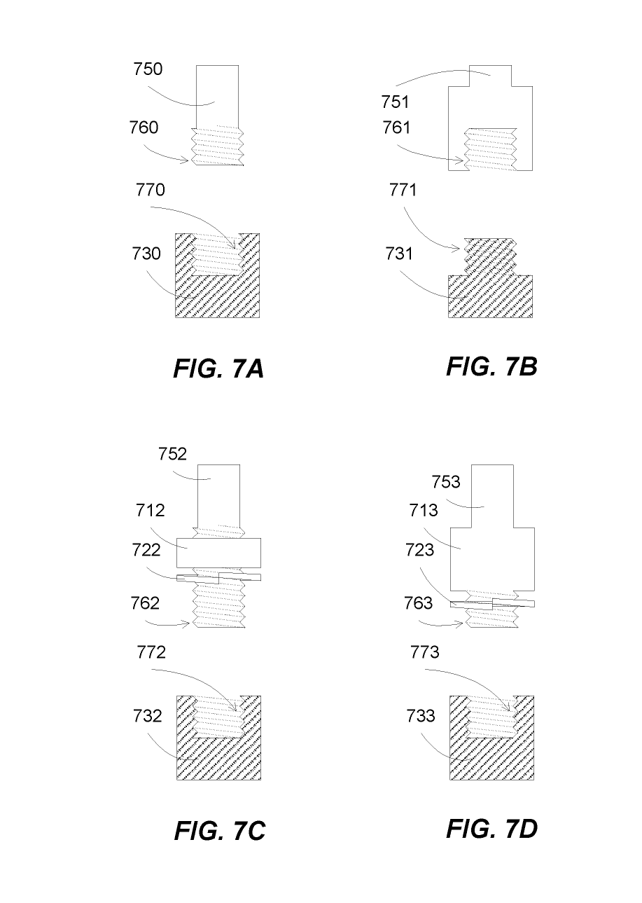

FIGS. 7A-7D illustrate configurations for secure connections between a plunger and a piston according to some embodiments. The secure connection can be a fixed connection, e.g., forming a rigid element of the plunger and the piston. The plunger and the piston can have mating thread, which can allow the plunger to be screwed in the piston. A locking mechanism, such as a lock washer, can be included.

FIG. 7A shows a thread connection between a plunger 750 and a piston 730. The plunger can have a male thread 760, and the piston can have a mating female thread 770. Thus the plunger can be screwed in the piston, forming a secure connection. A locking compound, such as a glue adhesive, can be coated the walls of the thread 760 or 770, which can provide a improved secure connection. In some embodiments, the depth of the female thread can be shorter than the length of the male thread, allowing a tight connection between the two components.

FIG. 7B shows a thread connection between a plunger 751 and a piston 731. The plunger can have a female thread 761, and the piston can have a mating male thread 771. Thus the plunger can be screwed in the piston, forming a secure connection. A locking compound, such as a glue adhesive, can be coated the walls of the thread 761 or 771, which can provide a improved secure connection. In some embodiments, the depth of the female thread can be shorter than the length of the male thread, allowing a tight connection between the two components.

FIG. 7C shows a secure connection between a plunger 752 and a piston 732. The plunger can have a male thread 762, and the piston can have a mating female thread 772. Thus the plunger can be screwed in the piston. A nut 712 and a lock washer 722 can be included, for form a better secure connection. Other configurations can be used, such as a regular washer instead of a lock washer. In the figures, a split lock washer is shown. Other lock washers can be used, such as star lock washers, toothed lock washers, serrated washers, cupped spring washers, conical washers, curved disc spring washers, wave washers or polymer washers such as a rubber washers. In addition, a locking compound can be used.

FIG. 7D shows a thread connection between a plunger 753 and a piston 733. The plunger can have a male thread 763, together with a stopping portion 713, and the piston can have a mating female thread 773. Thus the plunger can be screwed in the piston, forming a secure connection. A lock washer 723 can be included. In addition, a locking compound can be used.

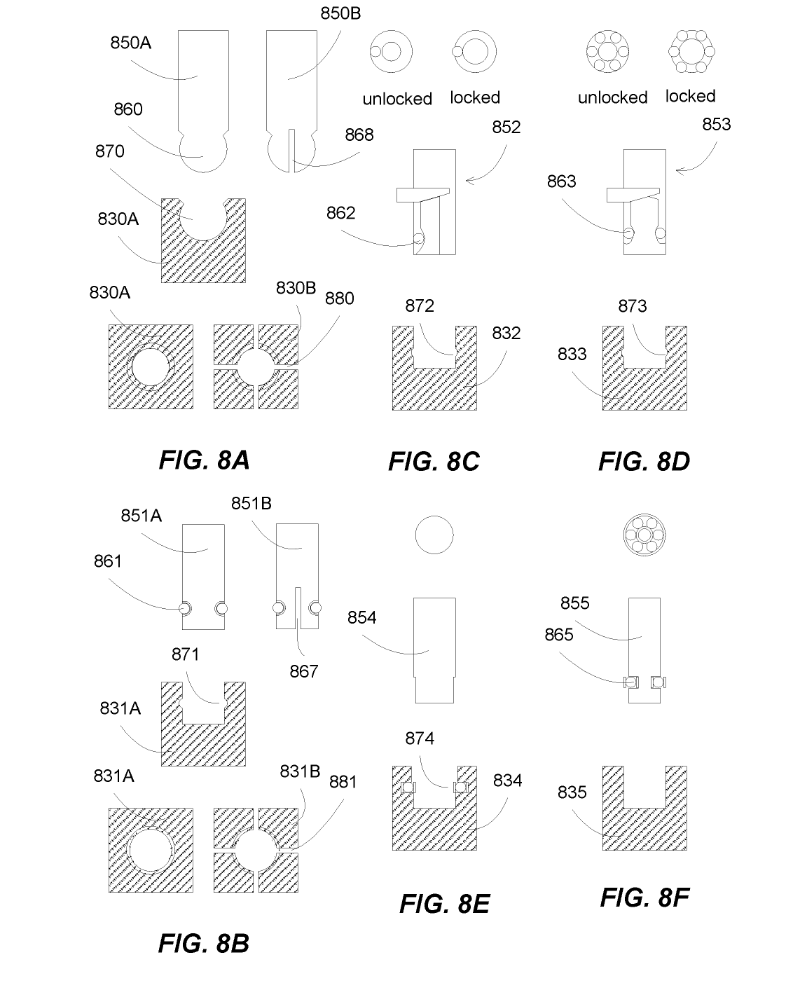

FIGS. 8A-8F illustrate linearly secure but rotatable connection configurations between a plunger and a piston according to some embodiments. The linearly secure connection configurations can prevent the plunger, after making the connection with the piston, from being removed from the piston when the plunger is exerting a pulling action, for example, to bring the material back to the split syringe barrel. The rotatable connection configurations can allow the plunger to rotate relative to the piston. The rotating action can be used to convert a rotating motion of the plunger to a linear motion of the piston, for example, using a translation mechanism such as a lead screw or a ball screw.

FIG. 8A shows a circular connection configuration, which can include a plunger 850A having a ball shape protrusion 860, which can be mated with a ball shape cavity 870 of a piston 830A. The ball shape can be modified, such as smoothing the opening of the cavity 870 to facilitate the insertion of the plunger 850A to the piston 830A. Alternatively, the ball shape protrusion can have splits 868, thus the ball shape protrusion 860 can be squeezed into the ball shape cavity 870. Alternatively, the piston 830B can have splits 880, for example, across the ball shape cavity portion, which then can allow the ball shape cavity to expand to accommodate the insertion of the ball shape protrusion 860. The ball shape mating can provide a secure and rotatable connection between the plunger and the piston.

FIG. 8B shows a cylindrical connection configuration, which can include a plunger 851A having a cylindrical shape, which can be mated with a cylindrical shape cavity of a piston 831A. The cylindrical shape of the plunger 851B can be modified, such as have splits 867, thus the cylindrical shape protrusion 851 can be squeezed into the cylindrical shape cavity. Alternatively, the piston 831B can have splits 881, for example, across the cylindrical shape cavity portion, which then can allow the cylindrical shape cavity to expand to accommodate the insertion of the cylindrical shape protrusion 851.

In addition, balls 861 can be disposed on the plunger 851A or 851B, for example, placed in recesses of the plunger body. The recesses can be configured so that the balls can rotate. In addition, the cylindrical cavity of the piston 831A or 831B can have a circular recess 871, which matches the shape of the plunger with the rotatable balls 861, e.g., matching the shape of the extended balls. Thus the plunger can be rotatable, while secured against being pulled out.

FIG. 8C shows a cylindrical connection configuration, which can include a plunger 852 having a cylindrical shape, together with a snap on mechanism that includes a ball 862. The ball 862 can be recessed into the cylindrical plunger, or can be extended outside the outer wall of the plunger, such as based on the snap on mechanism described above. In an unlocked configuration, the ball is recessed. In a locked configuration, the ball is extended.

The piston 832 can have a mating cylindrical cavity to accommodate the plunger. In addition, the cylindrical cavity can have a circular recess 872, which matches the shape of the plunger in the locked configuration, e.g., matching the shape of the extended ball. Thus the plunger can be rotatable, while secured against being pulled out.

FIG. 8D shows another cylindrical connection configuration, which can include a plunger 853 having a cylindrical shape, together with a snap on mechanism that includes multiple balls 863. The balls 863 can be recessed into the cylindrical plunger, or can be extended outside the outer wall of the plunger, such as based on the snap on mechanism described above. In an unlocked configuration, the ball is recessed. In a locked configuration, the ball is extended.

The piston 833 can have a mating cylindrical cavity to accommodate the plunger. In addition, the cylindrical cavity can have a circular recess 873, which matches the shape of the plunger in the locked configuration, e.g., matching the shape of the extended ball. Thus the plunger can be rotatable, while secured against being pulled out.

FIG. 8E shows a connection configuration, which can include a piston 834 having a bearing 874. The plunger 854 can have a circular cross section, which can press fit on the bearing, thus providing a secure and rotatable configuration.

FIG. 8F shows a connection configuration, which can include a plunger 855 having a bearing 875. The piston 835 can have a circular cross section, which can be pressed fit on the bearing of the plunger 855, thus providing a secure and rotatable configuration.

In some embodiments, the present invention discloses coupling configurations between a moving mechanism, such as lead screw or a ball screw mechanism, and a split syringe. The split syringe can include a piston having a coupling that does not provide rotating capability such as coupling 970. The coupling can provide a linearly secure connection, e.g., allowing a component to couple to the coupling without getting loose when the component is pushed in or pulled out with reasonable forces.

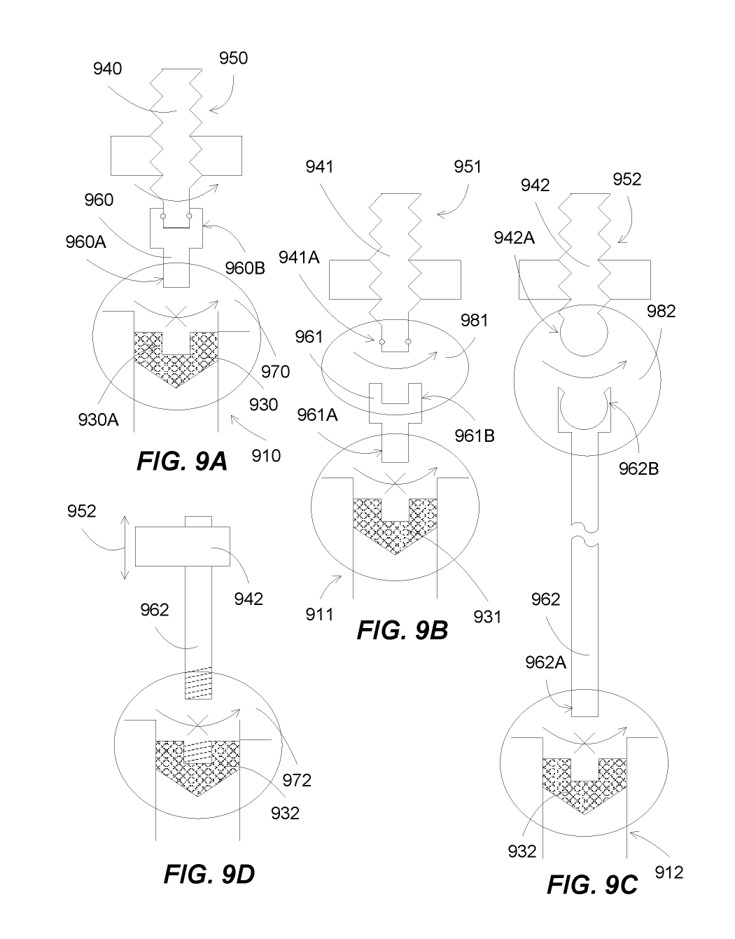

FIGS. 9A-9D illustrate coupling configurations between a moving mechanism and a split syringe according to some embodiments. The coupling configurations can include an integrated coupling, a separate coupling, and a separate long coupling. The coupling configurations can include a rotational free coupling (FIGS. 9A-9C) or a rigid coupling (FIG. 9D)

FIG. 9A shows an integrated coupling configuration. A coupling that allows a rotating action at one end, and keeping stationary at an opposite end can be integrated to a moving mechanism, such as integrated to an end of a rotating shaft of the moving mechanism. For example, the moving mechanism can include an integrated coupler that accepts a rotational action of the moving mechanism, while keeping the end, e.g., the portion that is coupled to the piston, stationary, for not disturbing, e.g., not rotating, the piston due to the rotating action of the moving mechanism. An integrated coupler 960 can be integrated to a moving mechanism 950, such as integrated to a rotatable lead screw 940 of a lead screw or ball screw mechanism. The integrated coupler 960 can include a rotation coupling at one end 960B, such as a bearing to allow the lead screw 940 to rotate against the stationary coupler 960. Thus the opposite end 960A of the coupler 960 can be stationary while the lead screw is rotating, which can allow the coupler 960 to couple to a non-rotatable coupler 930 of the split syringe 910. For example, the stationary portion 960A of the coupler 960 of the moving mechanism 950 can be coupled to the stationary portion 930A of the coupler 930 of the split syringe 910.

FIG. 9B shows an external coupling configuration. An external coupler 961 that allows a rotating action at one end, and keeping stationary at an opposite end can be coupled to a moving mechanism, such as couple to an end of a rotating shaft of the moving mechanism. For example, the moving mechanism can accept an external coupler that receives a rotational action of the moving mechanism, while keeping the end, e.g., the portion that is coupled to the piston, stationary, for not disturbing, e.g., not rotating, the piston due to the rotating action of the moving mechanism. An external coupler 961 can be coupled to a moving mechanism 951, such as coupling to a rotatable lead screw 941 of a lead screw or ball screw mechanism. The end 941A of the shaft 941 of the moving mechanism 951 can include a rotation coupling, such as a bearing, to allow the lead screw 941 to rotate against the stationary coupler 961, using the external coupler 961. The end of the external coupler 961 can include a mating element to mate with the bearing of the end 941A of the shaft 941. Thus the opposite end 961A of the coupler 961 can be stationary while the lead screw is rotating, which can allow the coupler 961 to couple to a non-rotatable coupler 931 of the split syringe 911. For example, the stationary portion 961A of the coupler 961 of the external coupler 961 can be coupled to the stationary portion 931A of the coupler 931 of the split syringe 911.

FIG. 9C shows an external long coupling configuration. An external long coupler 962 can be similar to the external coupling 961, with a difference of being long and optionally thin. The long coupler can be used for small split syringe, e.g., smaller than the size of the shaft of the moving mechanism, for allowing the long coupler to get inside the split syringe.

For example, the moving mechanism can accept an external long coupler that receives a rotational action of the moving mechanism, while keeping the end not rotating. An external long coupler 962 can be coupled to a moving mechanism 952, such as coupling to a rotatable lead screw 942 of a lead screw or ball screw mechanism. The end 942A of the shaft 942 of the moving mechanism 952 can include a rotation coupling, such as a ball shape coupler, to allow the lead screw 942 to rotate against the stationary coupler 962, using the external long coupler 962. The end of the external long coupler 962 can include a mating element to mate with the ball shape coupler of the end 942A of the shaft 942. Thus the opposite end 962A of the coupler 962 can be stationary while the lead screw is rotating, which can allow the coupler 962 to couple to a non-rotatable coupler 932 of the split syringe 912. For example, the stationary portion 962A of the coupler 962 of the external coupler 962 can be coupled to the stationary portion 932A of the coupler 932 of the split syringe 912.

FIG. 9D shows a fixed coupling configuration 972, e.g., a rigid coupling between the plunger 962 and the piston 932. The plunger 962 can be screwed in the piston 932, by a thread configuration. The coupling is fixed in all directions of movements, e.g., the plunger and the piston can move as one element.

In some embodiments, the plunger 962 can be coupled to a translation mechanism, such as a lead screw mechanism. For example, the plunger 962 can be fixedly coupled to a ball screw nut 942 of a lead screw mechanism. When a lead screw shaft of the lead screw mechanism rotates, the rotational movement can translate into a linear movement 952 of the lead screw nut, which linearly moves the plunger 962 and the piston 932.

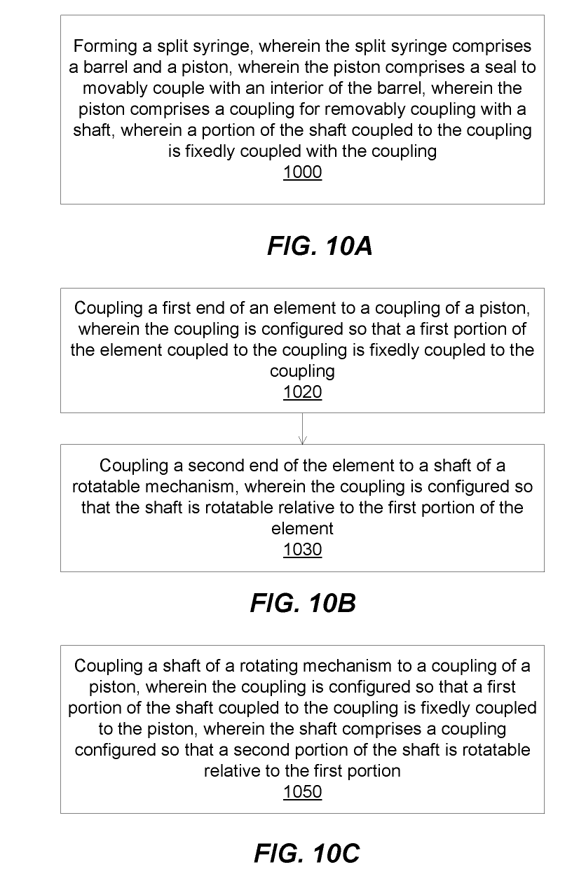

FIGS. 10A-10C illustrate flow charts for coupling a split syringe with a moving mechanism according to some embodiments. In FIG. 10A, a split syringe can be formed, which can allow filled split syringes to be transferred with small volumes as compared to filled conventional syringes, and with minimum potential damages as compared to filled conventional syringes, due to the lack of accidental pushing on the exposed plungers of the conventional syringes. Operation 1000 forms a split syringe, wherein the split syringe can include a barrel and a piston, wherein the piston can include a seal to movably couple with an interior of the barrel, wherein the piston can include a coupling for removably coupling with a shaft, wherein a portion of the shaft coupled to the coupling is fixedly coupled with the coupling.

In some embodiments, a split syringe can be formed. The split syringe can include a barrel, wherein the barrel can include a nozzle at one end, wherein the nozzle is configured to be coupled with a needle. The split syringe can include a piston disposed inside the barrel, such as partially or completely disposed in the barrel. The piston can be configured for a tight fitting in the barrel that allows retracting and expelling a material in the barrel through the nozzle. For example, the piston can include a seal for forming a seal fitting with the barrel.

In some embodiments, the piston can be configured to be removably coupled to a plunger. The plunger can be configured to be coupled to a translation mechanism. The translation mechanism translates a turning motion of a rotatable component into a linear motion of the piston. Thus a rotation motion of the translation mechanism can result in a linear motion of the piston, which can expel or retract material in the syringe. The translation mechanism can include a motor configured to turn the rotatable component, such as a lead screw shaft or the plunger itself.

In some embodiments, the piston can be configured to move linearly inside the barrel, for example, when a rotatable component of a translation mechanism can be rotating. The material can be configured to be expelled out of the barrel when the rotatable component rotates in one direction. The material can be configured to be retracted toward the barrel when the rotatable component rotates in an opposite direction.

In some embodiments, the piston can include a shaft, wherein the shaft can be short, such as shorter than half of the barrel. The shaft can be configured to be coupled to the plunger, e.g., the coupling of the piston with the plunger can be made at the shaft of the piston.

In some embodiments, the removable coupling between the piston and the plunger can be integrated in the piston, e.g., the piston can include a integrated coupled configuration that can form a removable coupling with the plunger. There can be a separate coupler that can be attached to the piston, so that the piston can be coupled to the plunger.

In some embodiments, the coupling of the nozzle with a needle can include a twisted and taper fitting for making a leak-free coupling with the needle, such as a Luer lock.

In some embodiments, the split syringe can further include a mounting assembly to support the barrel, wherein the mounting assembly can be configured to couple the barrel to a print head, wherein the print head can be configured to be mounted in a printer system. In some embodiments, the mounting assembly can be configured to couple the barrel to a 3D printer.

In some embodiments, the piston can be configured to be directly coupled to the plunger. The coupling between the plunger and the piston can be rigid in all directions, e.g., the plunger and the piston can move as one solid element in any direction. For example, the plunger can be configured to be pressed fit into the piston.

In some embodiments, the piston can include a thread for mating with the plunger. For example, the plunger can have a male thread for screwing into a female thread of the piston. The plunger can have a female thread for accepting a male thread of the piston.

The plunger/piston coupling can include a locking mechanism to lock the plunger with the piston. The locking mechanism can include a glue adhesive for securing the coupling between the plunger and the piston, such as using the glue adhesive on the thread of the thread coupling. The locking mechanism can include a lock washer for locking the plunger and the piston, especially if the plunger and the piston include a thread connection. The locking mechanism can include a latch mechanism to secure the plunger with the piston.

In some embodiments, the coupling between the plunger and the piston can be linearly rigid and rotation free. The linearly rigid configuration can restrict the plunger from being separated from the piston in a linear direction, such as in the directions of movement of the piston to expel and retract the material inside the syringe barrel. The directions of movement of the piston can be the directions along a length of the syringe barrel, e.g., from the flange end to the nozzle tip. The rotation free configuration can allow the plunger to rotate freely relative to the piston, e.g., either the plunger or the piston can rotate regardless of the motion of the other component.

In some embodiments, the coupling between the plunger and the piston can include a ball bearing assembly disposed between the piston and the plunger. The ball bearing assembly can be configured to allow the plunger to rotate relative to the piston. Alternatively, the plunger or the piston can include a ball assembly, which can also be coupled to the other component (e.g., the piston or the plunger, respectively). The ball assembly can be configured to allow the plunger to rotate relative to the piston. Alternatively, the coupling between the plunger and the piston can include a ball socket mechanism disposed between the piston and the plunger. The ball socket mechanism can be configured to allow the plunger to rotate relative to the piston.

In some embodiments, the plunger/piston coupling can include a latch mechanism. The latch mechanism can rigidly couple the plunger to the piston in a linear direction along a length of the barrel, while still allowing the plunger to rotate relative to the piston.

In some embodiments, at least one of the plunger and the piston can include a slit to assist in assembling of the piston with the plunger.

In some embodiments, the piston can be configured to be removably coupled to the plunger through an intermediate coupler, wherein at least one of the coupling between the plunger and the intermediate coupler and the coupling between the piston and the intermediate coupler can be rotatingly free. Both the couplings between the plunger and the intermediate coupler and between the piston and the intermediate coupler are linearly rigid, meaning when the plunger move toward or away from the syringe barrel, the piston also moves inward or outward of the syringe barrel. One coupling between the plunger and the intermediate coupler and between the piston and the intermediate coupler, e.g., either the coupling between the plunger and the intermediate coupler or the coupling between the piston and the intermediate coupler, can be rigid in all directions. The other coupling can be linearly rigid and rotation free.

A ball bearing assembly, a ball assembly, or a ball socket mechanism can be disposed between the piston and the intermediate coupler, or between the plunger and the intermediate coupler to provide a rotation free coupling.

A latch mechanism can be disposed between the piston and the intermediate coupler, or between the plunger and the intermediate coupler to provide a linearly rigid coupling, e.g., rigid coupling in a linear direction along a length of the barrel, while still allowing the plunger to rotate relative to the intermediate coupler, or the piston to rotate relative to the intermediate coupler.

In some embodiments, at least one of the element of the two elements of the couplings can include a slit to assist in assembling of the two elements together. For example, the plunger or one end of the intermediate coupler can include a slit to assist in the assembling of the plunger with the intermediate coupler. Also the piston or the other end of the intermediate coupler can include a slit to assist in the assembling of the piston with the intermediate coupler.

In some embodiments, the rotatable component of the translation mechanism can include the plunger, meaning the plunger can be the rotatable component of the translation mechanism. The coupling between the piston and the rotatable component can be a rotation free coupling, so that the piston can become the linear component of the translation mechanism, meaning the rotation of the plunger can translate into a linear motion of the piston.

In some embodiments, the plunger can be coupled to a linear component of the translation mechanism. The linear component can move linearly while a rotational component of the translation mechanism rotates. In other words, the translation mechanism can translate a rotational motion of the rotational component into a linear motion of the linear component. For example, for a translation mechanism that includes a lead screw, the rotational component can include the lead screw shaft, and the linear component can include the lead screw nut.

A coupling between the plunger and the piston can be a rigid coupling in all directions, since the rotation of the rotation component of the translation mechanism, such as the lead screw shaft, can be converted to a linear motion of the linear component, such as the lead screw nut. With the plunger coupled to the lead screw nut, the plunger can move linearly, pushing and pulling the piston in and out of the syringe barrel.

In FIG. 10B, a split syringe can be coupled to a moving mechanism through an intermediate coupler, which can be a short coupler or a long coupler. Operation 1020 couples a first end of an element to a coupling of a piston, wherein the coupling is configured so that a first portion of the element coupled to the coupling is fixedly coupled to the coupling. Operation 1030 couples a second end of the element to a shaft of a rotatable mechanism, wherein the coupling is configured so that the shaft is rotatable relative to the first portion of the element.

In FIG. 10C, a split syringe can be coupled to a moving mechanism through an integrated coupler. Operation 1050 couples a shaft of a rotating mechanism to a coupling of a piston, wherein the coupling is configured so that a first portion of the shaft coupled to the coupling is fixedly coupled to the piston, wherein the shaft can include a coupling configured so that a second portion of the shaft is rotatable relative to the first portion.

In some embodiments, the present invention discloses coupling configurations between a moving mechanism, such as lead screw or a ball screw mechanism, and a split syringe. The split syringe can include a piston having a coupling that provide rotating capability 1170. The coupling can provide a secure connection, e.g., allowing a component to couple to the coupling without getting loose when the component is pushed in or pulled out with reasonable forces. The coupling can further provide a rotating capability, e.g., allowing a component, after coupled with the coupling, to rotate without affecting the coupling, e.g., the component can rotate while the coupling stays stationary.

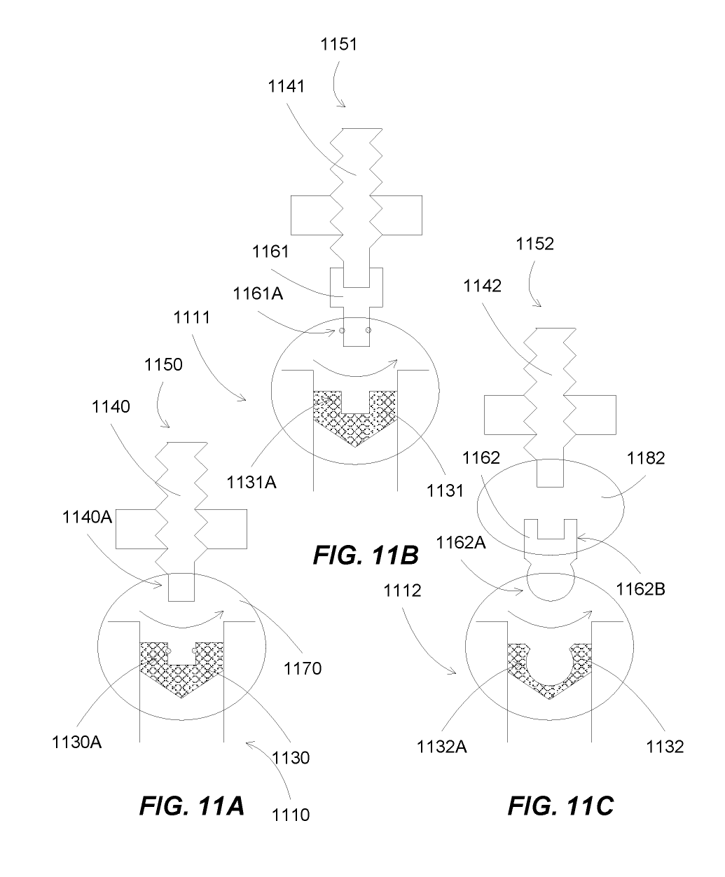

FIGS. 11A-11C illustrate coupling configurations between a moving mechanism and a split syringe according to some embodiments. The coupling configurations can include a direct coupling, an integrated coupling, and a separate coupling. Other couplings can also be used, such as separate long coupling for small split syringe applications.

FIG. 11A shows a direct coupling configuration. A moving mechanism can directly couple to the split syringe, e.g., a shaft end of the moving mechanism can be coupled to the coupling of the piston of the split syringe. This can be a simple connection, since the shaft of the moving mechanism can rotate while advancing forward or retracting backward, and the piston can accommodate the rotation and allowing the shaft to push or pull on the piston.

For example, the moving mechanism 1150 can include a rotating shaft 1140. The rotating shaft can be the screw of a lead screw or a ball screw mechanism, thus can rotate while moving linearly forward or backward depending on the rotational directions. The end 1140A of the shaft 1140 can be coupled directly to a piston 1130 of a split syringe 1110. Since the piston 1130 can accept a rotational movement, for example, due to rotating configuration 1130A such as a bearing, the shaft end 1140A can be connected directly to the piston. Other types of rotatable coupling can be used, such as the rotatable couplings disclosed above.

As shown, the rotating action is in the piston 1130, e.g., ball bearing 1130A can be installed in the piston 1130. The passive component of the rotating capability is in the shaft 1140 of the moving mechanism, e.g., a end portion 1140A of the shaft can be a solid piece having a cylindrical shape. Alternatively, the rotating action can be in the shaft end 1140A, e.g., bearing can be placed at the shaft end. The passive component can be the piston, e.g., the piston can have a cylindrical recess, which can accommodate the bearing in the shaft end.

FIG. 11B shows an integrated coupling configuration. The shaft 1141 of the moving mechanism 1151 can have a coupler 1161, which can be an integrated coupler to the shaft end. The coupler 1161 can have a bearing 1161A at an end, which can be coupled to the piston 1131 for rotating capability. The coupler 1131A of the piston 1131 can be a passive component, e.g., a cylindrical recess that can accommodate the bearing 1161A of the shaft end. Other types of rotatable coupling can be used, such as the rotatable couplings disclosed above.

As shown, the rotating action can be in the shaft end 1141A, e.g., bearing can be placed at the shaft end. The passive component can be the piston, e.g., the piston can have a cylindrical recess, which can accommodate the bearing in the shaft end. Alternatively, the rotating action is in the piston 1131, e.g., ball bearing 1131A can be installed in the piston 1131. The passive component of the rotating capability is in the shaft 1141 of the moving mechanism, e.g., a end portion 1141A of the shaft can be a solid piece having a cylindrical shape.

FIG. 11C shows an external coupling configuration. An external coupler 1162 that allows a rotating action at one end, and keeping stationary at an opposite end can be coupled to a moving mechanism, such as couple to an end of a rotating shaft of the moving mechanism. An external coupler 1162 can be coupled to a moving mechanism 1152, such as coupling to a rotatable lead screw 1142 of a lead screw or ball screw mechanism. The end of the shaft 1142 of the moving mechanism 1152 can be fixedly coupled to an end 1162B of the coupler 1162. The other end of the coupler 1162 can include a rotation coupling 1162A, such as a ball shape protrusion, to couple to a coupler 1132A of the piston 1132. Other types of rotatable coupling can be used, such as the rotatable couplings disclosed above.

The external coupler can be long, to accommodate small split syringe configurations. In addition, other combinations of couplings can be used, such as a combination of integrated coupler with an external coupler.

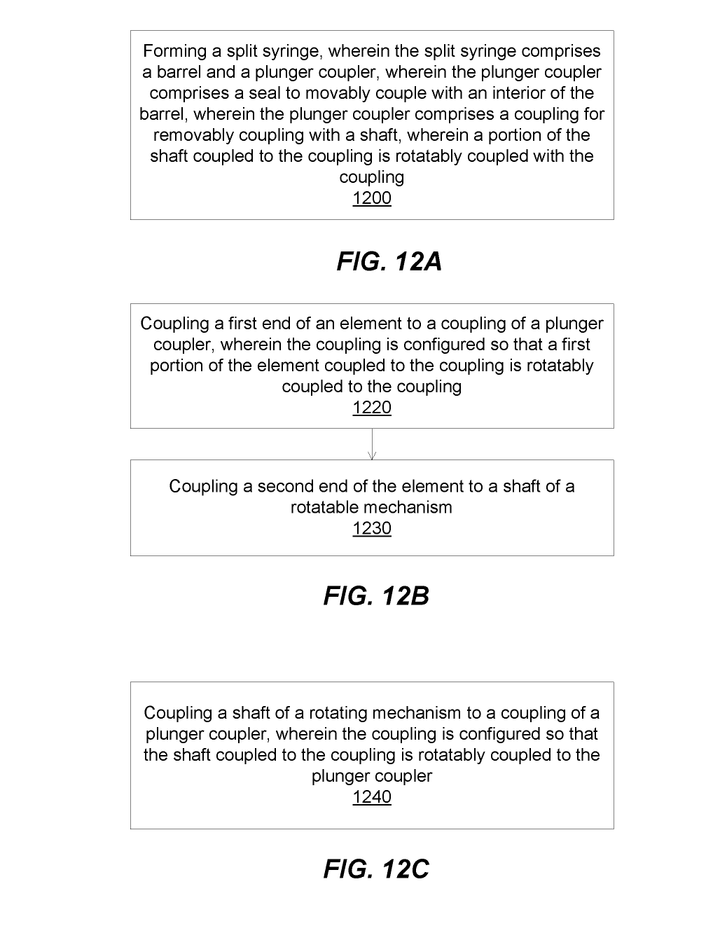

FIGS. 12A-12C illustrate flow charts for coupling a split syringe with a moving mechanism according to some embodiments. In FIG. 12A, a split syringe can be formed. Operation 1200 forms a split syringe, wherein the split syringe can include a barrel and a piston, wherein the piston can include a seal to movably couple with an interior of the barrel, wherein the piston can include a coupling for removably coupling with a shaft, wherein a portion of the shaft coupled to the coupling is rotatably coupled with the coupling.

In FIG. 12B, a split syringe can be coupled to a moving mechanism through an intermediate coupler, which can be a short coupler or a long coupler. Operation 1220 couples a first end of an element to a coupling of a piston, wherein the coupling is configured so that a first portion of the element coupled to the coupling is rotatably coupled to the coupling. Operation 1230 couples a second end of the element to a shaft of a rotatable mechanism.

In FIG. 12C, a split syringe can be coupled directly to a moving mechanism. Operation 1250 couples a shaft of a rotating mechanism to a coupling of a piston, wherein the coupling is configured so that the shaft coupled to the coupling is rotatably coupled to the piston.

In some embodiments, the present invention discloses methods, and syringes, such as split syringes, filling with materials resulted from the methods, that can fill a syringe with materials without voids or bubbles. Since the delivery of materials in the syringe can need to be accurately controlled, for example, in a printing process using a 3D printer, the rate of advance of the piston will be precisely controlled depending on, for example, the speed of the syringe during the printing process. Thus a void or a bubble in the syringe can result in a defect on the printed object.

To prevent bubbles or voids, careful filing of the material in the syringe barrel can be performed, to prevent the introduction of voids or bubbles when filling the barrel. In addition, especially for paste materials, voids or bubbles can already exist in the paste, such as in microscopic sizes. Thus a vacuum chamber treatment can be used to remove any voids or bubbles in the materials, before and after filling the syringe.

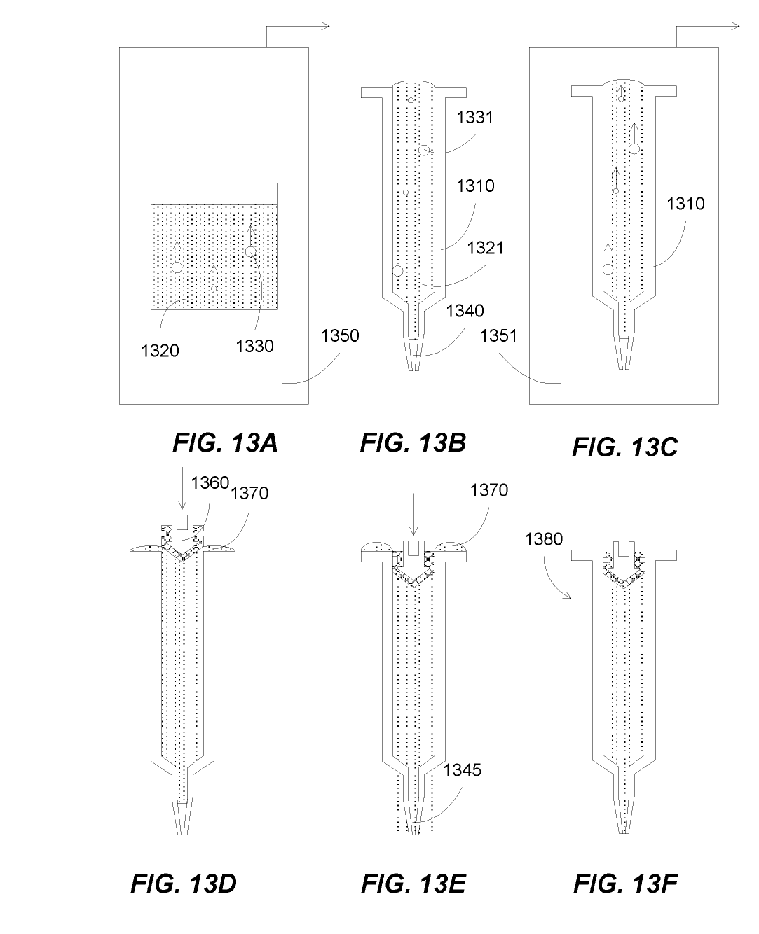

FIGS. 13A-13F illustrate a process of filling a syringe barrel for printing in a 3D printer according to some embodiments. In FIG. 13A, a material 1320 can be placed in a vacuum chamber 1350 for a time until the bubbles 1330 rise to the surface. In FIG. 13B, the de-bubble material 1321 can be placed in a syringe barrel 1310, for example, by pouring into an opening of the barrel. The material can fill the syringe barrel, even overfilled. Slow pouring can be used, to avoid bubble generation, such as by splashing or trapping. There can be a small area 1340 at the nozzle tip that does not have material. There can also be bubbles 1331 in the material 1321, for example, due to not careful pouring, due to bubble generation during pouring, or due to not completely removing bubbles in the previous bubble removal process in a vacuum chamber.

In FIG. 13C, an optional vacuum process can be performed, similar to the previous bubble removal process. The filled barrel 1310 can be placed in a vacuum chamber 1351, such as a vacuum chamber 1350 used in previous step. Vacuum can be applied, e.g., air in the chamber can be pumped out of the vacuum chamber to form a low pressure volume, e.g., lower than atmospheric pressure. The low pressure ambient can push the bubbles to the surface. If the material level drops, additional material can be added in for topping the syringe barrel.

In FIG. 13D, a piston 1360 can be placed at the opening of the syringe barrel. In FIG. 13E, the piston can be pushed into the barrel, spilling material 1370 out of the opening. The material should be filled in the barrel, so that there can be no trapped bubbles in the barrel when the piton completely enter the barrel. After the piston enters completely in the barrel, pressure in the barrel can push the material to the nozzle, filling empty space 1340 with material 1345. In FIG. 13F, a syringe barrel 1380 is filled with material without or with minimum bubbles.

As shown, the process is used to fill split syringe, e.g., the piston is shown without the plunger. This process can also be applied to conventional syringe, e.g., syringe with a piston/plunger assembly.

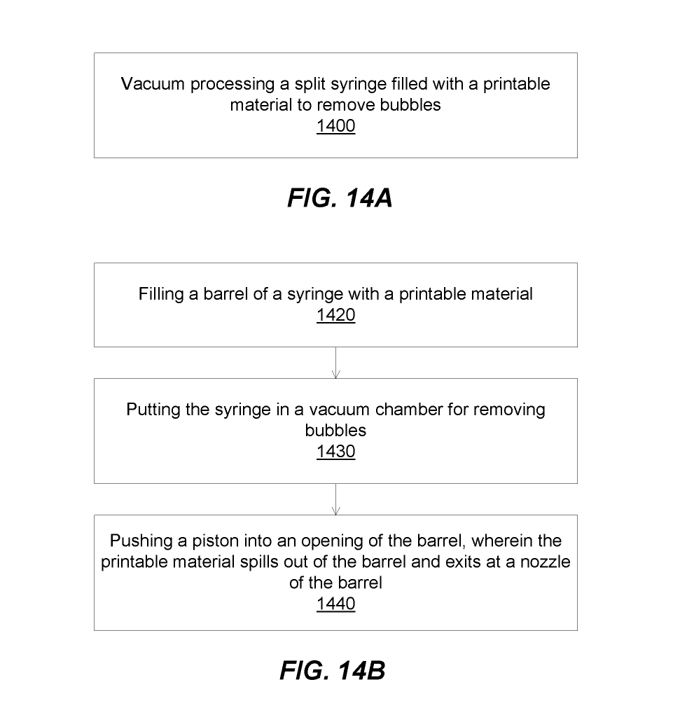

FIGS. 14A-14B illustrate flow charts for filling syringe barrels with materials according to some embodiments. In FIG. 14A, operation 1400 vacuum processing materials before or after filling a syringe barrel, wherein the material is a printable material for used in a 3D printing process.

In FIG. 14B, operation 1420 fills a barrel of a syringe with a printable material, wherein the material is optionally vacuum processed to remove bubbles. Operation 1420 optionally puts the syringe in a vacuum chamber for removing bubbles. Operation 1420 pushes a piston into an opening of the barrel, wherein the printable material spills out of the barrel and exits at a nozzle of the barrel.

In some embodiments, the present invention discloses print heads that are configured to accept split syringes for printing the materials stored in the split syringes. For example, the print head can include a support for holding the barrel of the split syringe. The print head can be mounted on a printer, such as a 3D printer, or the print head can include an electrical and mechanical interface for removably coupling with a printer. The print head can include a moving mechanism, such as a translation mechanism, which is configured to drive a shaft forward and optionally backward. The shaft can connect, e.g., couple, with the split syringe, and the shaft can act as a plunger for moving a piston (or plunger seal) of the split syringe.

In some embodiments, the moving mechanism can include a motor driving a translation mechanism such as a lead screw (including a ball screw) mechanism. The lead screw mechanism can include a rotatable shaft, rotating under the actuating action of the motor. The rotation motion of the rotatable shaft can be translated into a linear motion, such as through a lead screw nut. The linear motion can drive a plunger/piston assembly downward to deliver the material in the split syringe, or upward to pull the material back into the split syringe.

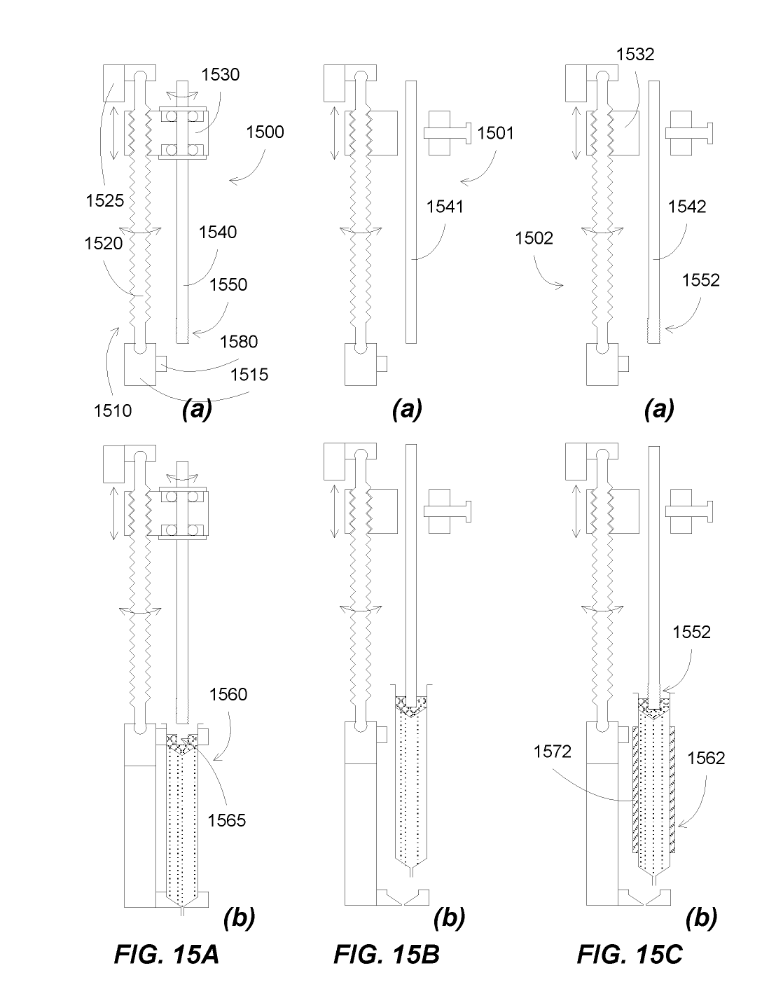

FIGS. 15A-15C illustrate print head configurations according to some embodiments. FIG. 15A (a) shows a print head 1500 having a lead screw mechanism 1510, e.g., a translation mechanism, which includes a rotatable lead screw shaft 1520, which is coupled to a lead screw nut 1530. The coupling of the lead screw nut to the lead screw shaft is such that the rotation motion of the lead screw shaft can translate into a linear motion of the lead screw nut. A mechanical and electrical interface 1525 can be coupled to the lead screw mechanism, to couple the print head to a 3D printer. A mounting assembly 1580 can be coupled to the lead screw mechanism, to couple the print head to a split syringe. A motor 1515 can be coupled to the lead screw shaft, to rotate the lead screw shaft.

The lead screw nut 1530 can include a linearly rigid and rotation free coupling to a plunger shaft 1540. The plunger shaft can be rigidly coupled to the lead screw nut in the direction of linear motion of the lead screw nut, e.g., the up/down directions as shown in the figure. The plunger shaft can be rotatably coupled to the lead screw nut, for example, by using ball bearings. The plunger shaft can include thread 1550 at one end of the shaft, which can be configured to mate with a piston of a split syringe.

FIG. 15A(b) shows an assembling process to couple a split syringe into the print head 1500. A split syringe 1560 can be mounted to the print head, e.g., through the mounting assembly 1580. The plunger shaft 1540 can be lower to contact the split syringe, for example, by rotating the lead screw shaft. Alternatively, the lead screw shaft can be freely rotatable, for example, due to the free running state of the motor, and the plunger shaft can be pushed down to contact the split syringe, forcing the lead screw shaft to rotate.

The plunger shaft can rotate, to screw into the mating thread 1565 of the piston. A locking mechanism, such as a lock washer, can be included, to secure the plunger shaft to the piston. Thus when the lead screw shaft rotates, the lead screw nut moves in a linear direction, pushing in or pulling out the piston with respect to the syringe barrel.

FIGS. 15B(a) and 15B(b) show a print head configuration 1501, in which the plunger can be fixed coupled to the lead screw nut. The plunger 1541 can be fixedly coupled to the split syringe, and then the plunger/split syringe assembly can be fixedly coupled to the lead screw nut.

FIGS. 15C(a) and 15C(b) show a print head configuration 1502, in which the plunger can be fixed coupled to the lead screw nut, for example, by a thread mechanism 1552. The plunger 1542 can be threaded to the piston of the split syringe, and then the plunger/split syringe assembly can be fixedly coupled to the lead screw nut. A heater assembly 1572 can be coupled to the split syringe 1562 to heat the material inside the split syringe.

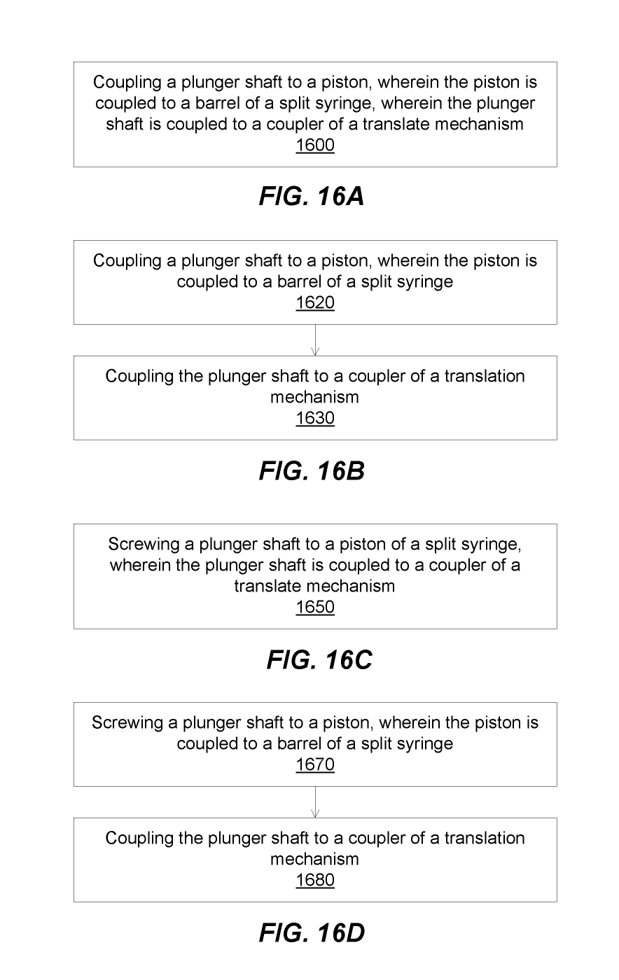

FIGS. 16A-16D illustrate flow charts for forming print heads according to some embodiments. In FIG. 16A, operation 1600 couples a plunger shaft to a piston, wherein the piston is coupled to a barrel of a split syringe, wherein the plunger shaft is coupled to a coupler of a translate mechanism.

In FIG. 16B, operation 1620 couples a plunger shaft to a piston, wherein the piston is coupled to a barrel of a split syringe. Operation 1630 couples the plunger shaft to a coupler of a translation mechanism

In FIG. 16C, operation 1650 screws a plunger shaft to a piston of a split syringe, wherein the plunger shaft is coupled to a coupler of a translate mechanism.

In FIG. 16D, operation 1670 screws a plunger shaft to a piston, wherein the piston is coupled to a barrel of a split syringe. Operation 1680 couples the plunger shaft to a coupler of a translation mechanism.

In some embodiments, the moving mechanism can include a motor driving a lead screw (including a ball screw) mechanism. The lead screw mechanism can include a rotatable shaft, rotating under the actuating action of the motor. The rotatable shaft can function as the plunger, or can be coupled to a plunger shaft.

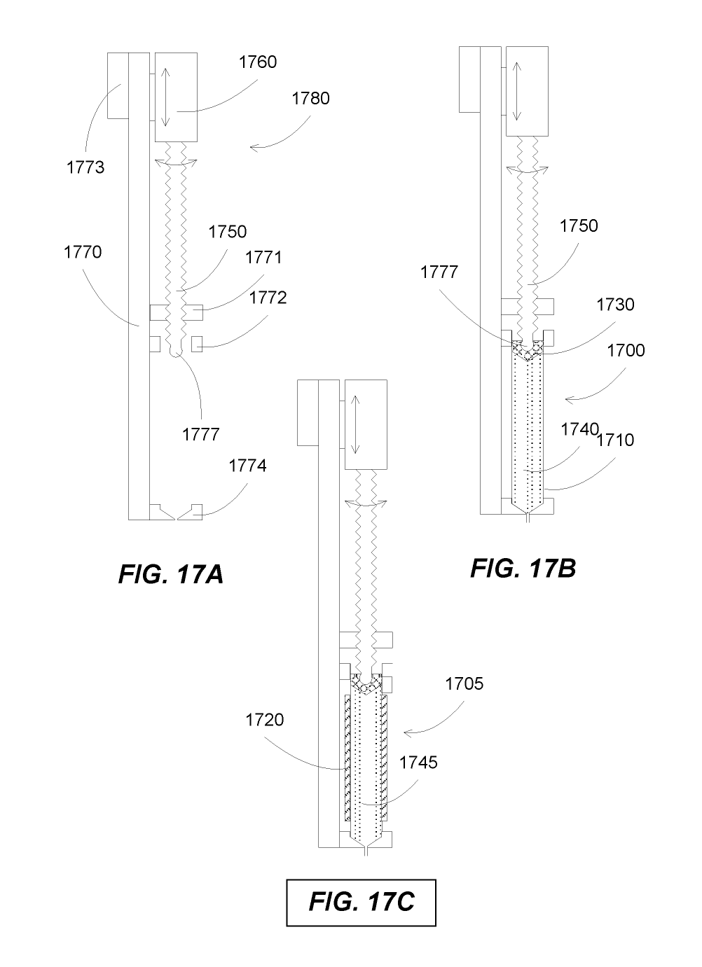

FIGS. 17A-17C illustrate print head configurations according to some embodiments. In FIG. 17A, a print head 1780 can include a moving mechanism that can include a motor 1760 that can drive a shaft 1750. The motor and the shaft can move relative to a stationary support 1770 that includes a block 1771 in which the shaft is rotated. An interface 1773 can be coupled to the stationary support 1770, and the interface can be used to coupled to a printer, such as a 3D printer. A support 1772/1774 can be coupled to the stationary support 1770 to support a split syringe. The shaft 1750 can include a coupling 1777 for coupling with a piston of the split syringe.

FIG. 17B shows the print head assembly together with a split syringe 1700 mounted on the print head assembly. The split syringe can include a barrel 1710 which is configured to hold a material 1740, for example, a 3D printing material. The split syringe can include a piston 1730, which can be coupled to the shaft 1750 of the moving mechanism in the print head assembly 1780, for example, through a rotatable coupling 1777. Other coupling configurations can be used, such as the couplings disclosed above.

FIG. 17C shows the print head assembly with a heated split syringe 1705. The split syringe can include a heater assembly 1720, such as a heater jacket surrounding the syringe. The split syringe can be filled with a material 1745 that might need to be heated before printing. For example, the print material 1745 can be a solid at room temperature. Thus by heating, the material 1745 can become a semi-liquid or a liquid, which can allow a delivery by the split syringe.

In some embodiments, the present invention discloses print heads that are configured to accept conventional syringes, e.g., syringe having a piston coupled to a plunger, for printing the materials stored in the syringes. For example, the print head can include a support for holding the barrel of the syringe. The print head can be mounted on a printer, such as a 3D printer, or the print head can include an electrical and mechanical interface for removably coupling with a printer. The print head can include a moving mechanism, such as a translation mechanism, which is configured to drive a component forward and optionally backward. The component can connect, e.g., couple, with the syringe, e.g., with the plunger of the conventional syringe.

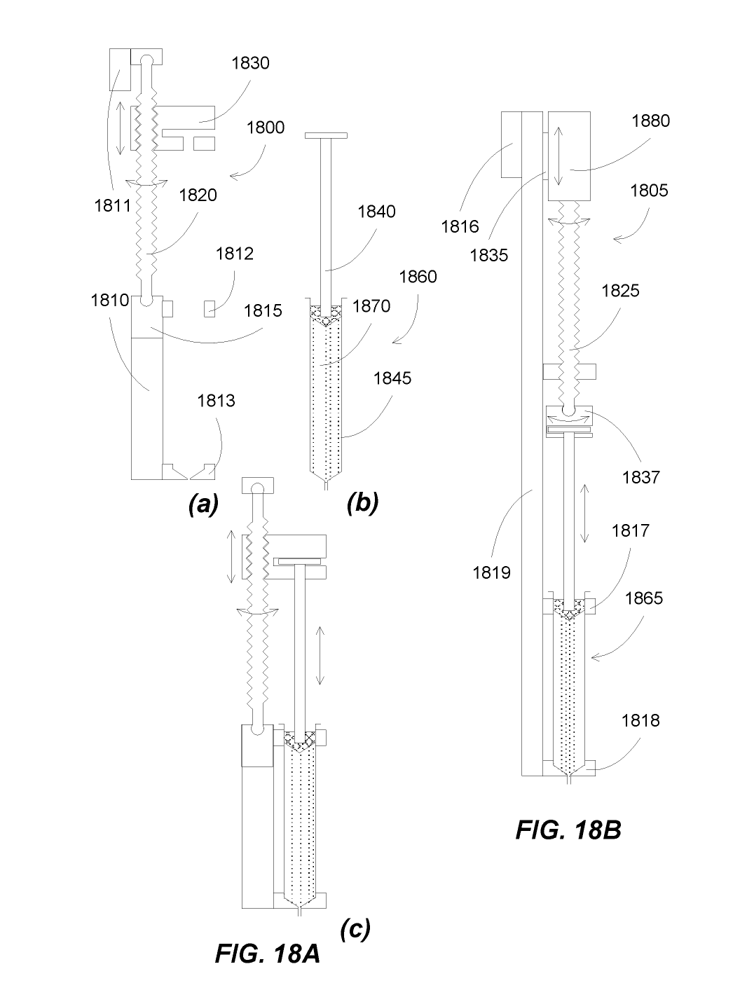

FIGS. 18A-18B illustrate print head configurations according to some embodiments. In some embodiments, the moving mechanism can include a motor driving a translation mechanism such as a lead screw (including a ball screw) mechanism. The lead screw mechanism can include a rotatable shaft, rotating under the actuating action of the motor. The rotation motion of the rotatable shaft can be translated into a linear motion, such as through a lead screw nut. The linear motion can drive a plunger of a conventional syringe downward to deliver the material in the syringe, or upward to pull the material back into the syringe.

FIGS. 18A (a)-18A(c) show a print head 1800, which can be configured to accept a conventional syringe 1860. The conventional syringe can include a syringe barrel 1845 and a plunger 1840 coupled to a piston. The syringe can be filled with a printing material 1870. The syringe can be filled with the material according to a method described above to minimizing bubbles.

A print head 1800 can include a lead screw mechanism 1810, e.g., a translation mechanism, which includes a rotatable lead screw shaft 1820, which is coupled to a lead screw nut 1830. The coupling of the lead screw nut to the lead screw shaft is such that the rotation motion of the lead screw shaft can translate into a linear motion of the lead screw nut. A mechanical and electrical interface 1811 can be coupled to the lead screw mechanism, to couple the print head to a 3D printer. A mounting assembly 1812/1813 can be coupled to the lead screw mechanism, to couple the print head to a syringe. A motor 1815 can be coupled to the lead screw shaft, to rotate the lead screw shaft.

The lead screw nut 1830 can include a mounting configuration for coupling to an end of a plunger shaft 1840 of a syringe 1860. The plunger shaft can be rigidly coupled to the lead screw nut.

The syringe 1860 can be coupled to the mounting assembly 1812/1813 with the plunger coupled to the lead screw nut 1830. Thus when the lead screw shaft rotates, the lead screw nut moves in a linear direction, pushing in or pulling out the piston with respect to the syringe barrel.

In some embodiments, the moving mechanism can include a motor driving a lead screw (including a ball screw) mechanism. The lead screw mechanism can include a rotatable shaft, rotating under the actuating action of the motor. The rotatable shaft can coupled to a plunger shaft of a conventional syringe. The coupling between the rotatable shaft and the plunger can be a linearly fixed coupling and rotation free coupling.

FIG. 18B shows a print head 1805, which can be configured to accept a conventional syringe 1865. The print head 1805 can include a moving mechanism that can include a motor 1880 that can drive a shaft 1825. The motor and the shaft can move relative to a stationary support 181. An electrical and mechanical interface 1816 can be coupled to the stationary support 181, and the interface can be used to coupled to a printer, such as a 3D printer. A support 1817/1818 can be coupled to the stationary support 1819 to support a syringe 1865. The shaft 1825 can include a coupling 1837 for coupling with a plunger end of the syringe.

When the motor runs, the shaft 1825 also rotates. The rotation motion of the rotatable shaft 1825 can be translated to a linear motion of the coupling 1837. The coupling 1837 can drive the plunger into or out of the barrel of the syringe.

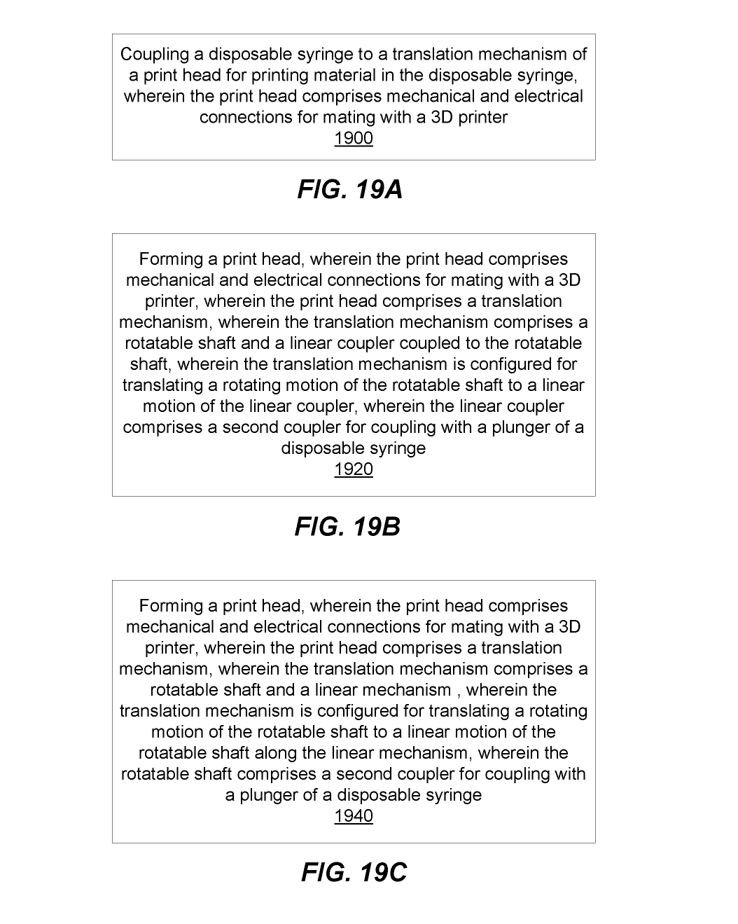

FIGS. 19A-19C illustrate flow charts for forming print heads according to some embodiments. In FIG. 19A, operation 1900 couples a disposable syringe to a translation mechanism of a print head for printing material in the disposable syringe, wherein the print head comprises mechanical and electrical connections for mating with a 3D printer. In FIG. 19B, operation 1920 forms a print head, wherein the print head comprises mechanical and electrical connections for mating with a 3D printer, wherein the print head comprises a translation mechanism, wherein the translation mechanism comprises a rotatable shaft and a linear coupler coupled to the rotatable shaft, wherein the translation mechanism is configured for translating a rotating motion of the rotatable shaft to a linear motion of the linear coupler, wherein the linear coupler comprises a second coupler for coupling with a plunger of a disposable syringe. In FIG. 19C, operation 1940 forms a print head, wherein the print head comprises mechanical and electrical connections for mating with a 3D printer, wherein the print head comprises a translation mechanism, wherein the translation mechanism comprises a rotatable shaft and a linear mechanism, wherein the translation mechanism is configured for translating a rotating motion of the rotatable shaft to a linear motion of the rotatable shaft along the linear mechanism, wherein the rotatable shaft comprises a second coupler for coupling with a plunger of a disposable syringe.

In some embodiments, the present invention discloses printers, such as 3D printers, that can be configured to accept print heads having split syringes for printing the materials stored in the split syringes. For example, the printer can have an interface for mounting one or more print heads. One print head can be configured to accept a split syringe and printing materials stored in the split syringe. One such print head has been disclosed above.

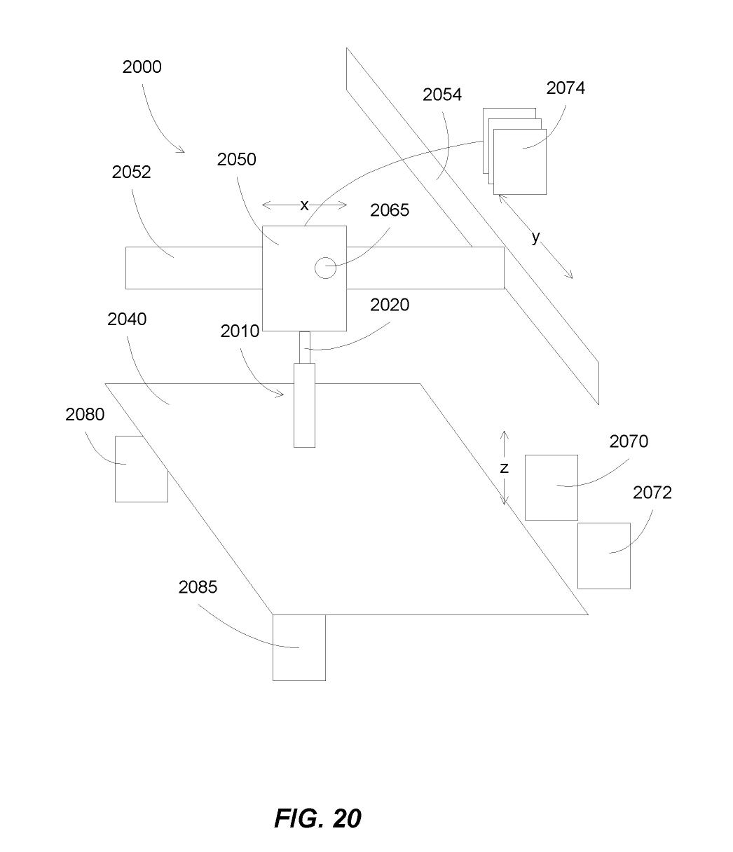

FIG. 20 illustrates a schematic of a printer for split syringe usage according to some embodiments. The printer 2000 can include a print head 2050. The print head can be integrated to the printer. The print head can be removably attached to the printer. That way, different print heads can be coupled to a same printer. For example, one print head can be removed from the printer, and another print head can be attached to the printer. The printer can be configured to accept multiple print heads, thus can have any combination of the multiple print heads.

A print head can be configured to accept a split syringe 2010. The print head can be a print head among the multiple print heads that can be removably attached to the printer, or the print head that is integrated to the printer. For example, the print head can include a moving mechanism that includes a lead screw or plunger-acting component 2020, for coupling with a piston in the split screw for driving the materials in the split syringe. Different configurations of print head can be used, including the print head that has been described above.

The printer 2000 can include a platform 2040 for supporting a printed object. The platform 2040 can move in a z direction, for example, up and down, to bring the platform 2040 closer to a print head 2050. In some embodiments, the platform can move in a z direction, for example, up and down, to print objects along the z direction. The platform can move relative to the print head, for example, to bring the printed objects closer to the print head, the platform can move up or the print head can move down. A z mechanism 2070 can be used to control the z movement of the platform or the print head. For example, the z mechanism can be coupled to the platform to move the platform up and down. Alternatively, the z mechanism can be coupled to the print head, e.g., to an assembly that includes the print head, to move the print head up and down. A distance sensor 2065 can be coupled to the printer head, or to the printer head assembly, e.g., to the mechanism that moves the printer head. The distance sensor can be configured for sensing a distance from the printer head to the platform. For example, the distance sensor can be a laser sensor or an ultrasonic sensor. The sensor can be used for calibrating the platform with respect to the print head.

In some embodiments, an antivibration or damping mechanism 2080 can be included. The antivibration or damping mechanism can be coupled to the platform to reduce vibration, for example, caused by movements of the moving mechanism, such as movements of the platform or movements of the print head assembly. The antivibration or damping mechanism can pacify the platform, allowing a flat and stationary surface for ease of printing.

In some embodiments, a heater 2085 can be included. The heater can be coupled to the platform to heat the platform. Alternatively, the heater can be coupled to the printer to provide a heated environment. For example, the printer can have an enclosure, and the heater can be placed inside the enclosure to heat the interior of the enclosure.

The printer head 2050 can move in lateral directions, such as x and y directions, or r and theta directions. For example, a moving mechanism 2052 can be configured to move the printer head 2050 in the x direction. A moving mechanism 2054 can be configured to move the printer head assembly, e.g., the print head and the moving mechanism 2052, in the y direction. Other moving mechanisms can be used, such as an x-y table configured to move the printer head. In addition, the platform can be stationary, with the printer head moves in the z direction. A controller 2072 can be included to move the printer head according to a pattern for printing on the platform. Other components can be included, such as additional print heads and filaments 2074 for filament printing print heads.

In some embodiments, an operator can install, on a printer, a print head that can accept a split syringe. The split syringe can already installed on the print head. Alternatively, the split syringe can be installed on the print head after the print head has been installed on the printer. The printer can automatically configured to accept the newly installed print head, or the operator can manually set up the configuration.

In some embodiments, the 3D printer can be configured to accept a print head that is configured to accept a conventional syringe. Also, the 3D printer can included a print head that is configured to accept a conventional syringe.

Other components can be added, such as a heater assembly for heating the syringe.

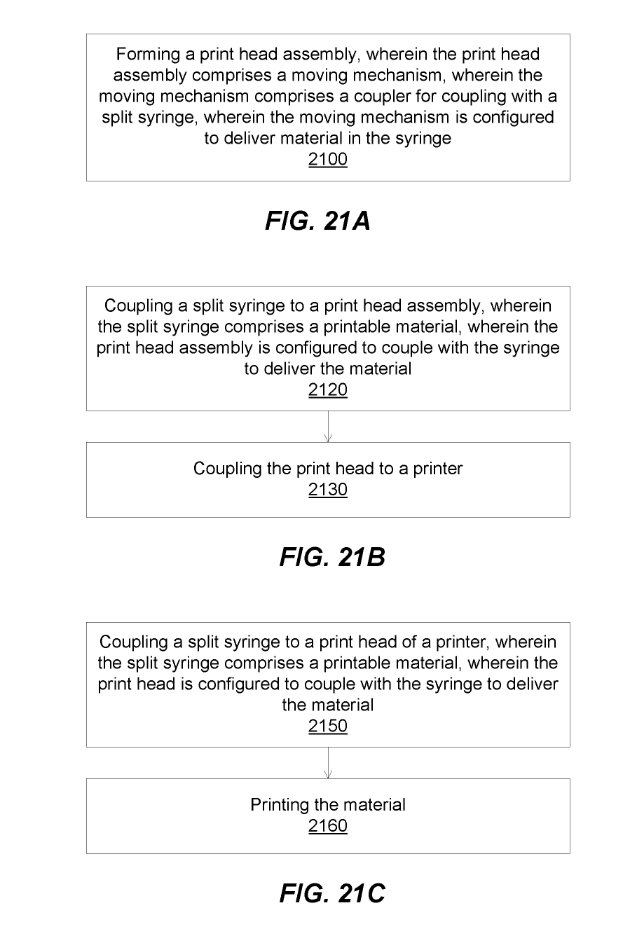

FIGS. 21A-21C illustrate flow charts for split syringes in print head or printer systems according to some embodiments. FIG. 21A shows a method to form a print head assembly that can be configured to accept a split syringe. Operation 2100 forms a print head assembly, wherein the print head assembly can include a moving mechanism, wherein the moving mechanism can include a coupler for coupling with a split syringe, wherein the moving mechanism is configured to deliver material in the syringe.

FIG. 21B shows a method to form a printer that can use a split syringe, for example, through a print head assembly. Operation 2120 couples a split syringe to a print head assembly, wherein the split syringe can include a printable material, wherein the print head assembly is configured to couple with the syringe to deliver the material. Operation 2130 couples the print head to a printer.

FIG. 21C shows a method to print from a printer that have a split syringe installed. Operation 2150 couples a split syringe to a print head of a printer, wherein the split syringe can include a printable material, wherein the print head is configured to couple with the syringe to deliver the material. Operation 2160 prints the material.

What is claimed is:

1. A split syringe comprising
a barrel, wherein the barrel comprises a nozzle at one end, wherein the nozzle is configured to be coupled with a needle,
a piston disposed inside the barrel, wherein the piston is configured for a tight fitting in the barrel that allows retracting and expelling a material in the barrel through the nozzle,
wherein the piston is configured to be removably coupled to a plunger,
wherein the plunger is configured to be coupled to a translation mechanism,
wherein the translation mechanism translates a turning motion of a rotatable component into a linear motion of the piston.

2. A split syringe as in claim 1
wherein the piston comprises a shaft, wherein the shaft is shorter than the half the barrel.

3. A split syringe as in claim 1
wherein the coupling of the nozzle comprises a Luer lock.

4. A split syringe as in claim 1 further comprising
a mounting assembly to support the barrel, wherein the mounting assembly is configured to couple the barrel to a print head, wherein the print head is configured to be mounted in a printer system.

5. A split syringe as in claim 1 further comprising
a mounting assembly to support the barrel, wherein the mounting assembly is configured to couple the barrel to a 3D printer.

6. A split syringe as in claim 1
wherein the translation mechanism comprises a first coupler, wherein the first coupler is coupled to the rotatable component, wherein the first coupler is coupled to the plunger.

7. A split syringe as in claim 1
wherein the translation mechanism comprises a lead screw mechanism with the rotatable component being the lead screw shaft.

8. A split syringe as in claim 1
wherein the piston comprises a thread for mating with the plunger.

9. A split syringe as in claim 1
wherein the plunger is rotatably coupled to a first coupler, wherein the first coupler is coupled to the rotatable component.

10. A split syringe as in claim 1
wherein the translation mechanism comprises a lead screw mechanism with the rotatable component being the lead screw shaft,
wherein the plunger is rotatably coupled to a lead screw nut, wherein the lead screw nut is coupled to the lead screw shaft, wherein one end of the plunger comprises a thread,
wherein the piston comprises a mating thread to mate with the thread of the plunger.

11. A split syringe as in claim 1
wherein the rotatable component comprises the plunger, wherein the piston is configured to be directly coupled to the plunger, wherein the coupling between the piston and the plunger is rotatingly free.

12. A split syringe as in claim 1
wherein the rotatable component comprises the plunger, wherein the piston is configured to be coupled to the plunger through a second coupler, wherein at least one of the coupling between the plunger and the second coupler and the coupling between the piston and the second coupler is rotatingly free.

13. A print head comprising
a translation mechanism, wherein the translation mechanism translates a turning motion of a first component into a linear motion of second component,
a first mounting assembly for electrically and mechanically attaching to a movement mechanism of a 3D printer, wherein the first mounting assembly is coupled to the translation mechanism,
a second mounting assembly for accepting a pump assembly, wherein the pump assembly comprises a piston and a barrel, wherein the piston is configured for a tight fitting in the barrel that allows retracting and expelling a material in the barrel through a nozzle, wherein the first mounting assembly is coupled to the translation mechanism,

14. A print head as in claim 13
wherein the pump assembly comprises a disposable syringe.

15. A print head as in claim 13
wherein the pump assembly comprises a split syringe, wherein the split syringe does not comprise a plunger.

16. A print head as in claim 13
wherein the translation mechanism comprises a lead screw mechanism,
wherein the first component comprises a lead screw shaft,
wherein the second component comprises a lead screw nut, wherein the lead screw nut is coupled to the lead screw shaft,
wherein the lead screw nut is rotatably coupled to a plunger, wherein one end of the plunger comprises a thread,
wherein the piston comprises a mating thread to mate with the thread of the plunger.

17. A print head as in claim 13
wherein the first component comprises a rotatable plunger,
wherein the second component comprises the piston, wherein the plunger is rotatably coupled to the piston.

18. A 3D printer comprising
a platform defining a volume above the platform,
a movement mechanism,
a print head mounting assembly coupled to the movement mechanism, wherein the print head mounting assembly is configured to accept a print head for printing an object on the platform, wherein the print head is configured to accept a pump assembly, wherein the pump assembly comprises a piston and a barrel, wherein the piston is configured for a tight fitting in the barrel that allows retracting and expelling a material in the barrel through a nozzle
wherein the movement mechanism is configured to move the print head mounting assembly in the volume above the platform.

19. A 3D printer as in claim 18
wherein the pump assembly comprises a disposable syringe.

20. A 3D printer as in claim 18
wherein the pump assembly comprises a split syringe, wherein the split syringe does not comprise a plunger.
